(19) United States
(12) Patent Application Publication
(10) Pub. No.: US 2011/0086850 A1
(43) Pub. Date: Apr. 14, 2011

(54) RADIOSENSITIZATION OF TUMORS WITH INDAZOLPYRROLOTRIAZINES FOR RADIOTHERAPY

(75) Inventors: Kie-Kian Ang, Houston, TX (US); Luka Milas, Houston, TX (US)

(73) Assignee: Board of Regents, The University of Texas System, Austin, TX (US)

(21) Appl. No.: 12/937,241

(22) PCT Filed: Apr. 10, 2009
(86) PCT No.: PCT/US09/02255

§ 371 Date: Dec. 20, 2010

Related U.S. Application Data
(60) Provisional application No. 61/044,436, filed on Apr. 11, 2008.

(51) Int. Cl. A61K 31/5355; C12N 5/071; A61K 31/53; A61P 35/00

(52) U.S. Cl. 514/233.2; 435/325; 514/243

(57) ABSTRACT

Provided herein is a method of radiosensitizing a tumor in a subject, which comprises administering to the subject a radiosensitizing amount of an indazolpyrrolotriazine of Formula I. Also provided herein is a method of treating a tumor in a subject, which comprises the steps of administering to the subject a radiosensitizing amount of an indazolpyrrolotriazine of Formula I, and a therapeutically effective fraction of radiation.

### CROSS REFERENCE TO RELATED APPLICATION

This application claims the benefit of the priority of U.S. Provisional Application No. 61/044,436, filed Apr. 11, 2008, the disclosure of which is incorporated herein by reference in its entirety.

### FIELD

Provided herein is a method of radiosensitizing a tumor in a subject, which comprises administering to the subject a radiosensitizing amount of an indazolpyrrolotriazine. Also provided herein is a method of treating a tumor in a subject, which comprises administering to the subject a radiosensitizing amount of an indazolpyrrolotriazine and a therapeutically effective dose of radiation.

### BACKGROUND

Cancer is a major public health problem in the United States and other developed countries. Currently, cancer is the second leading cause of death in the United States. One in four deaths in the United States is caused by cancer (Jemal, et al., CA Cancer J. Clin. 2005, 55, 10-30). Radiotherapy is one of the most common cancer treatments. In the United States, about 70% of cancer patients receive radiotherapy as part of their cancer treatment. Radiotherapy can be used to treat localized solid tumors, such as cancers of the skin, tongue, larynx, brain, breast, and uterine cervix. It can also be used to treat leukemia and lymphoma. One major limitation of radiotherapy, however, is that tumors can develop resistance to it.

It has been reported that radiation activates EGFR signaling leading to radiation resistance (Schmidt-Ullrich, et al., Int. J. Radiat. Oncol. Biol. Phys. 1994, 29, 813-819; Schmidt-Ullrich, et al., Radiat Res. 1996, 145, 81-85; Contessa, et al., Clin. Cancer Res. 1999, 5, 405-411; Dent, et al., Mol. Biol. Cell. 1999, 10, 4231-4246; Lammering, et al., Clin. Cancer Res. 2001, 7, 682-690; Liang, et al., Int. J. Radiat. Oncol. Biol. Phys. 2003, 57, 246-254; and Ochs, Int. J. Radiat. Oncol. Biol. Phys. 2004, 58, 941-949). It has also been reported that the levels of EGFR expression are correlated with the resistance of the tumors to radiotherapy (Akimoto, et al., Clin. Cancer Res. 1999, 5, 2884-2890). Despite these advances in understanding molecular and cellular bases for radioresistance, there exists a long-felt need for effective therapies for cancer treatment, especially for those that are resistant to conventional therapies, including radiotherapy.

### SUMMARY OF THE DISCLOSURE

Provided herein is a method of radiosensitizing a tumor in a subject, comprising administering to the subject a radiosensitizing amount of an indazolpyrrolotriazine of Formula I:

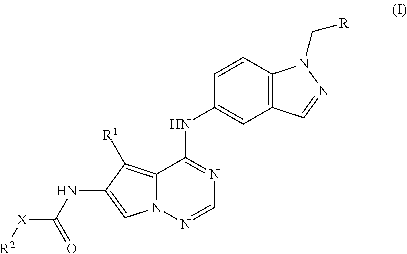

or an enantiomer, a mixture of enantiomers, or a mixture of two or more diastereomers thereof; or a pharmaceutically acceptable salt, solvate, hydrate, or prodrug thereof; wherein:
R is C₆₋₁₄ aryl or heterocyclyl;
R¹ is C₁₋₆ alkyl;
R² is hydrogen, C₁₋₆ alkyl, C₃₋₁₀ cycloalkyl, C₆₋₁₄ aryl, C₇₋₂₀ aralkyl, or heterocyclyl;
X is a bond, O, S, C(R³R⁴), or NR³; and
each R³ and R⁴ is independently hydrogen, C₁₋₆ alkyl, C₆₋₁₄ aryl, C₇₋₂₀ aralkyl, or heterocyclyl;
wherein each alkyl, cycloalkyl, aryl, aralkyl, and heterocyclyl is optionally substituted with one or more substituents Q selected from the group consisting of (a) cyano, halo, and nitro; (b) C₁₋₆ alkyl, C₂₋₆ alkenyl, C₂₋₆ alkynyl, C₃₋₁₀ cycloalkyl, C₆₋₁₄ aryl, heteroaryl, and heterocyclyl; and (c) —C(O)R^{a}, —C(O)OR^{a}, —C(O)NR^{b}R^{c}, —C(═NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{a}R^{d}, —NR^{a}C(O)R^{b}, —NR^{a}C(O)OR^{b}, —NR^{a}C(O)NR^{b}R^{c}, —NR^{a}C(═NR^{d})NR^{b}R^{c}, —NR^{a}S(O)R^{b}, —NR^{a}S(O)₂R^{b}, —NR^{a}S(O)NR^{b}R^{c}, —NR^{a}S(O)₂NR^{b}R^{c}, —SR^{a}, —S(O)R^{a}, and —S(O)₂R^{a}; wherein each R^{a}, R^{b}, R^{c}, and R^{d} is independently (i) hydrogen, C₁₋₆ alkyl, C₂₋₆ alkenyl, C₂₋₆ alkynyl, C₃₋₁₀ cycloalkyl, C₆₋₁₄ aryl, heteroaryl, or heterocyclyl; or (ii) R^{b} and R^{c} together with the N atom to which they are attached form heterocyclyl or heteroaryl.

Also provided herein is a method of treating a tumor in a subject, comprising the steps of: (A) administering to the subject a radiosensitizing amount of an indazolpyrrolotriazine of Formula I as described herein; and (B) administering to the subject a therapeutically effective dose of radiation.

Further provided herein is a method of radiosensitizing a cell, comprising contacting the cell with a radiosensitizing amount of an indazolpyrrolotriazine of Formula I as described herein.

Additionally, provided herein is a method of inhibiting the growth of a cell, comprising the steps of: (A) contacting the cell with a radiosensitizing amount of an indazolpyrrolotriazine of Formula I as described herein; and (B) exposing the cell to an effective dose of radiation sufficient to inhibit the cell growth.

### BRIEF DESCRIPTION OF THE DRAWINGS

FIG. 1 shows the effect of an indazolpyrrolotriazine of Formula I, AC480, on HN-5 cell growth.

FIG. 2 shows the effect of an indazolpyrrolotriazine of Formula I, AC480, on HNSCC-10 cell growth

FIG. 3 shows the effect of an indazolpyrrolotriazine of Formula I, AC480, on the radiosensitivity of HN-50 cells treated with AC480 at 300 nM (gray circle) and 600 nM (solid circle) prior to irradiation, along with the control (open circle).

FIG. 4 shows the effect of an indazolpyrrolotriazine of Formula I, AC480, on the radiosensitivity of HNSCC-10 cells treated with AC480 at 300 nM (gray circle) and 600 nM (solid circle) prior to irradiation, along with the control (open circle).

FIG. 5 shows the effect of an indazolpyrrolotriazine of Formula I, AC480, on radiation-induced apoptosis in HN-5 cells.

FIG. 6 shows the effect of an indazolpyrrolotriazine of Formula I, AC480, on radiation-induced accumulation of HN-5 cells in the G1, S, and G2 cell cycle phases.

FIG. 7 shows the effect of an indazolpyrrolotriazine of Formula I, AC480, at 600 nM (solid circle, dashed line) on the prolongation of γ-H2AX foci as assessed by immunocytochemistry 24 hrs after radiation. The control is shown as open circle and solid line.

FIG. 8 shows the effect of an indazolpyrrolotriazine of Formula I, AC480, on the radiosensitivity of HN-5 tumors in a xenograft mice model. Control is shown as solid circle and solid square, the treatment with AC480 alone is shown as solid triangle, the treatment with radiation alone is shown as open circle and open square, and the treatment with both AC480 and radiation is shown as open triangle.

### DETAILED DESCRIPTION

To facilitate understanding of the disclosure set forth herein, a number of terms are defined below.

Generally, the nomenclature used herein and the laboratory procedures in chemistry, biochemistry, biology, pharmacology, radiology, and others described herein are those well known and commonly employed in the art. Unless defined otherwise, all technical and scientific terms used herein generally have the same meaning as commonly understood by one of ordinary skill in the art to which this disclosure belongs.

The term “tumor,” “neoplasm,” and “neoplastic disorder or disease” are used interchangeably herein and are meant to refer to unwanted cell proliferation of one or more subset of cells in a multicellular organism resulting in harm (i.e., discomfort or decreased life expectancy) to the multicellular organisms. In certain embodiments, a tumor can be benign (non-invasive) or malignant (invasive).

The term “cancer” is meant to refer to a malignant neoplasm, which is characterized by uncontrolled cell proliferation where cells have lost their normal regulatory controls that would otherwise govern the rate of cell growth. These unregulated, dividing cells can spread throughout the body and invade normal tissues in a process referred to as “metastasis.”

The term “naturally occurring” or “native” when used in connection with biological materials such as nucleic acid molecules, polypeptides, host cells, and the like, refers to materials which are found in nature and are not manipulated by man. Similarly, “non-naturally occurring” or “non-native” refers to a material that is not found in nature or that has been structurally modified or synthesized by man.

The terms “HER1,” “epidermal growth factor receptor,” “EGFR,” and “ErbB1” are used interchangeably herein and refer to an EGFR receptor protein or variant thereof, as described, for example, in Carpenter et al., Ann. Rev. Biochem. 1987, 56, 881-914. HER2 variants include proteins substantially homologous to a native EGFR, i.e., proteins having one or more naturally or non-naturally occurring amino acid deletions, insertions or substitutions (e.g., EGFR derivatives, homologs and fragments), as compared to the amino acid sequence of a native EGFR. The amino acid sequence of an HER2 variant is at least about 80% identical, at least about 90% identical, or at least about 95% identical to a native EGFR. An example of naturally occurring mutant forms of a native EGFR, i.e. a deletion mutant EGFR, is described in Humphrey et al., Proc. Natl. Acad. Sci. USA 1990, 87, 4207-4211.

The terms “HER2” and “ErbB2” are used interchangeably herein and refer to a HER2 receptor protein or variant thereof. For example, a human HER2 protein is described in Semba et al., Proc. Natl. Acad. Sci. USA 1985, 82, 6497-6501 and Yamamoto et al. Nature 1986, 319, 230-234 (Genebank accession number X03363). HER2 variants include proteins substantially homologous to a native HER2, i.e., proteins having one or more naturally or non-naturally occurring amino acid deletions, insertions or substitutions (e.g., HER2 derivatives, homologs and fragments), as compared to the amino acid sequence of a native HER2. The amino acid sequence of a HER2 variant is at least about 80% identical, at least about 90% identical, or at least about 95% identical to a native HER2.

The term “overexpress” or “overexpression” is meant that a cell associated with a disease, disorder, or condition comprises a detectably higher level of a protein, such as HER1 or HER2, than an otherwise identical cell that is not associated with a disease, disorder or condition.

The term “subject” refers to an animal, including, but not limited to, a mammal, including a primate (e.g., human), cow, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The term “contacting” or “contact” is meant to refer to bringing together of a therapeutic agent and cell or tissue such that a physiological and/or chemical effect takes place as a result of such contact. Contacting can take place in vitro, ex vivo, or in vivo. In one embodiment, a therapeutic agent is contacted with a cell in cell culture (in vitro) to determine the effect of the therapeutic agent on the cell. In another embodiment, the contacting of a therapeutic agent with a cell or tissue includes the administration of a therapeutic agent to a subject having the cell or tissue to be contacted.

The terms “dose enhancement ratio” and “dose enhancement factor” are used interchangeably herein and are used herein to measure the fold increase in cytotoxic efficacy of radiation when delivered in the presence of a radiosensitizer. In certain embodiments, dose enhancement ratio is calculated by dividing the radiation dose required to achieve a surviving fraction in the absence of the radiosensitizer with the radiation dose required to achieve the same surviving fraction in the presence of the radiosensitizer at a specified concentration. This calculation method can be used to assess the radiosensitizing effect of an radiosensitizer in an in vitro system, such as cells in culture. In certain embodiments, dose enhancement ratio is calculated by dividing normalized growth delay in subject(s), such as mice, treated with a radiosensitizer and radiation by absolute tumor growth delay in subject(s) of the same species but treated with radiation alone, wherein the absolute tumor growth delay in subject(s) treated with radiation alone is defined as the difference between the time in days for tumor(s) in the subject(s) treated with radiation to grow from a first specified size to a second specified size, such as from 7 mm to 12 mm for mice, and the time in days for tumor(s) in untreated subject(s) of the same species to grow from the first specified size to the second specified size; and normalized growth delay in subject(s) treated with a radiosensitizer and radiation is defined as the difference between the time in days for tumor(s) in subject(s) treated with both the radiosensitizer and radiation to grow from the first specified size to the second specified size and the time in days for tumor(s) in subject(s) of the same species treated with the radiosensitizer alone to grow from the first specified size to the second specified size. See, Dote et al., Clin. Cancer Res. 2005, 11, 4571-4579; and Wiednmann et al, Clin. Cancer Res. 2007, 13, 1868-1874.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The term “alkyl” refers to a linear or branched saturated monovalent hydrocarbon radical, wherein the alkyl may optionally be substituted as described herein. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms), n-propyl, isopropyl, butyl (including all isomeric forms), n-butyl, isobutyl, sec-butyl, t-butyl, pentyl (including all isomeric forms), and hexyl (including all isomeric forms). For example, C₁₋₆ alkyl refers to a linear saturated monovalent hydrocarbon radical of 1 to 6 carbon atoms or a branched saturated monovalent hydrocarbon radical of 3 to 6 carbon atoms.

The term “alkenyl” refers to a linear or branched monovalent hydrocarbon radical, which contains one or more, in one embodiment, one to five, in another embodiment, one, carbon-carbon double bond(s). The alkenyl may be optionally substituted as described herein. The term “alkenyl” also embraces radicals having “cis” and “trans” configurations, or alternatively, “Z” and “E” configurations, as appreciated by those of ordinary skill in the art. As used herein, the term “alkenyl” encompasses both linear and branched alkenyl, unless otherwise specified. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propen-1-yl, propen-2-yl, allyl, butenyl, and 4-methylbutenyl.

The term “alkynyl” refers to a linear or branched monovalent hydrocarbon radical, which contains one or more, in one embodiment, one to five, in another embodiment, one, carbon-carbon triple bond(s). The alkynyl may be optionally substituted as described herein. The term “alkynyl” also encompasses both linear and branched alkynyl, unless otherwise specified. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkynyl groups include, but are not limited to, ethynyl (—C≡CH) and propargyl (—CH₂C≡CH). For example, C₂₋₆ alkynyl refers to a linear unsaturated monovalent hydrocarbon radical of 2 to 6 carbon atoms or a branched unsaturated monovalent hydrocarbon radical of 3 to 6 carbon atoms.

The term “cycloalkyl” refers to a cyclic monovalent hydrocarbon radical, which may be optionally substituted as described herein. In one embodiment, cycloalkyl groups may be saturated, and/or bridged, and/or non-bridged, and/or fused bicyclic groups. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkyl groups include, but are not limited to, cyclopropyl, cyclobutyl, cyclopentyl, cyclohexyl, cycloheptyl, decalinyl, bicyclo[2.2.1]heptanyl, and adamantyl.

The term “aryl” refers to a monovalent monocyclic aromatic group and/or monovalent multicyclic aromatic group that contain at least one aromatic carbon ring. In certain embodiments, the aryl has from 6 to 20 (C₆₋₂₀), from 6 to 15 (C₆₋₁₅), or from 6 to 10 (C₆₋₁₀) ring atoms. Examples of aryl groups include, but are not limited to, phenyl, naphthyl, fluorenyl, azulenyl, anthryl, phenanthryl, pyrenyl, biphenyl, and terphenyl. Aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl(tetralinyl). In certain embodiments, aryl may be optionally substituted as described herein.

The term “aralkyl” or “arylalkyl” refers to alkyl substituted with one or more aryl groups. In certain embodiments, the aralkyl has from 7 to 30 (C₇₋₃₀), from 7 to 20 (C₇₋₂₀), or from 7 to 16 (C₇₋₁₆) carbon atoms. Examples of aralkyl groups include, but are not limited to, benzyl, 2-phenylethyl, and 3-phenylpropyl. In certain embodiments, aralkyl may also be optionally substituted with one or more substituents as described herein.

The term “heteroaryl” refers to a monovalent monocyclic aromatic group and/or multicyclic aromatic group that contain at least one aromatic ring, wherein at least one aromatic ring contains one or more heteroatoms independently selected from O, S, and N in the ring. Heteroaryl groups are bonded to the rest of the molecule through the aromatic ring. Each ring of a heteroaryl group can contain one or two O atoms, one or two S atoms, and/or one to four N atoms, provided that the total number of heteroatoms in each ring is four or less and each ring contains at least one carbon atom. In certain embodiments, the heteroaryl has from 5 to 20, from 5 to 15, or from 5 to 10 ring atoms. Examples of monocyclic heteroaryl groups include, but are not limited to, furanyl, imidazolyl, isothiazolyl, isoxazolyl, oxadiazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, heteroaryl may also be optionally substituted as described herein.

The term “heterocyclyl” or “heterocyclic” refers to a monovalent monocyclic non-aromatic ring system and/or multicyclic ring system that contains at least one non-aromatic ring, wherein one or more of the non-aromatic ring atoms are heteroatoms independently selected from O, S, or N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. Heterocyclyl groups are bonded to the rest of the molecule through the non-aromatic ring. In certain embodiments, the heterocyclyl is a monocyclic, bicyclic, tricyclic, or tetracyclic ring system, which may include a fused or bridged ring system, and in which the nitrogen or sulfur atoms may be optionally oxidized, the nitrogen atoms may be optionally quaternized, and some rings may be partially or fully saturated, or aromatic. The heterocyclyl may be attached to the main structure at any heteroatom or carbon atom which results in the creation of a stable compound. Examples of such heterocyclic radicals include, but are not limited to, azepinyl, benzodioxanyl, benzodioxolyl, benzofuranonyl, benzopyranonyl, benzopyranyl, benzotetrahydrofuranyl, benzotetrahydrothienyl, benzothiopyranyl, benzoxazinyl, β-carbolinyl, chromanyl, chromonyl, cinnolinyl, coumarinyl, decahydroisoquinolinyl, dihydrobenzisothiazinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isobenzotetrahydrofuranyl, isobenzotetrahydrothienyl, isochromanyl, isocoumarinyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. In certain embodiments, heterocyclic may also be optionally substituted as described herein.

The term “halogen”, “halide” or “halo” refers to fluorine, chlorine, bromine, and/or iodine.

The term “optionally substituted” is intended to mean that a group, such as an alkyl, alkenyl, alkynyl, cycloalkyl, aryl, heteroaryl, heterocyclyl, or alkoxy group, may be substituted with one or more substituents independently selected from, e.g., (a) alkyl, alkenyl, alkynyl, cycloalkyl, aryl, heteroaryl, and heterocyclyl, each optionally substituted with one or more, in one embodiment, one, two, three, or four, substituents Q; and (b) halo, cyano (—CN), nitro (—NO₂), —C(O)R^{a}, —C(O)OR^{a}, —C(O)NR^{h}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, heteroaryl, or heterocyclyl, each optionally substituted with one or more, in one embodiment, one, two, three, or four, substituents Q; or (iii) R^{b} and R^{c} together with the N atom to which they are attached form heterocyclyl, optionally substituted with one or more, in one embodiment, one, two, three, or four, substituents Q. As used herein, all groups that can be substituted are “optionally substituted,” unless otherwise specified.

In one embodiment, each Q is independently selected from the group consisting of (a) cyano, halo, and nitro; and (b) C₁₋₆ alkyl, C₂₋₆ alkenyl, C₂₋₆ alkynyl, C₃₋₇ cycloalkyl, C₆₋₁₄ aryl, heteroaryl, and heterocyclyl; and —C(O)R^{e}, —C(O)OR^{e}, —C(O)NR^{f}R^{g}, —C(NR^{e})NR^{f}R^{g}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S (O)NR^{f}R^{g}, —NR^{e}S (O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₇ cycloalkyl, C₆₋₁₄ aryl, heteroaryl, or heterocyclyl; or (iii) R^{f} and R^{g} together with the N atom to which they are attached form heterocyclyl.

In certain embodiments, “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of one enantiomer and about 5% or less of the other enantiomer based on the total weight of the racemate in question.

In describing an optically active compound, the prefixes R and S are used to denote the absolute configuration of a molecule about its chiral center(s). The (+) and (−) are used to denote the optical rotation of a compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the molecule, R and S.

The term “solvate” refers to a compound provided herein or a salt thereof, which further includes a stoichiometric or non-stoichiometric amount of solvent bound by non-covalent intermolecular forces. Where the solvent is water, the solvate is a hydrate.

### Radiosensitizing Agents

Compounds suitable for use as radiosensitizing agents in the methods provided herein include indazolylpyrrolotriazines of Formula I:

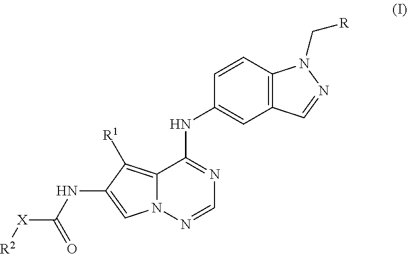

and enantiomers, mixtures of enantiomers, and mixtures of two or more diastereomers thereof; and pharmaceutically acceptable salts, solvates, hydrates, and prodrugs thereof; wherein:
R is C₆₋₁₄ aryl or heterocyclyl;
R¹ is C₁₋₆ alkyl;
R² is hydrogen, C₁₋₆ alkyl, C₃₋₁₀ cycloalkyl, C₆₋₁₄ aryl, C₇₋₂₀ aralkyl, or heterocyclyl;
X is a bond, O, S, C(R³R⁴), or NR³; and
each R³ and R⁴ is independently hydrogen, C₁₋₆ alkyl, C₆₋₁₄ aryl, C₇₋₂₀ aralkyl, or heterocyclyl;
wherein each alkyl, cycloalkyl, aryl, aralkyl, and heterocyclyl is optionally substituted with one or more substituents Q selected from the group consisting of (a) cyano, halo, and nitro; (b) C₁₋₆ alkyl, C₂₋₆ alkenyl, C₂₋₆ alkynyl, C₃₋₁₀ cycloalkyl, C₆₋₁₄ aryl, heteroaryl, and heterocyclyl; and (c) —C(O)R^{a}, —C(O)OR^{a}, —C(O)NR^{b}R^{c}, —C(═NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{a}R^{d}, —NR^{a}C(O)R^{b}, —NR^{a}C(O)OR^{b}, —NR^{a}C(O)NR^{b}R^{c}, —NR^{a}C(═NR^{d})NR^{b}R^{c}, —NR^{a}S(O)R^{b}, —NR^{a}S(O)₂R^{b}, —NR^{a}S(O)NR^{b}R^{c}, —NR^{a}S(O)₂NR^{b}R^{c}, —SR^{a}, —S(O)R^{a}, and —S(O)₂R^{a}; wherein each R^{a}, R^{b}, R^{c}, and R^{d} is independently (i) hydrogen, C₁₋₆ alkyl, C₂₋₆ alkenyl, C₂₋₆ alkynyl, C₃₋₁₀ cycloalkyl, C₆₋₁₄ aryl, heteroaryl, or heterocyclyl; or (ii) R^{b} and R^{c} together with the N atom to which they are attached form heterocyclyl or heteroaryl.

In one embodiment, R is C₆₋₁₄ aryl, R¹ is C₁₋₄ alkyl, each optionally substituted with one or more substituents Q, in one embodiment, one, two, or three substituents Q.

In another embodiment, X is O, and R² is C₃₋₁₀ cycloalkyl or heterocyclyl, each optionally substituted with one or more substituents Q, in one embodiment, one, two, or three substituents Q.

In yet another embodiment, the indazolylpyrrolotriazine provided herein is selected from the group consisting of:
[5-ethyl-4-[[(1-phenylmethyl)-1H-indazol-5-yl]amino]pyrrolo[2,1-f][1,2,4]triazin-6-yl]-carbamic acid, (3S)-3-morpholinylmethyl ester;
[5-ethyl-4-[[(1-phenylmethyl)-1H-indazol-5-yl]amino]pyrrolo[2,1-f][1,2,4]triazin-6-yl]-carbamic acid, (2R)-2-pyrrolidinylmethyl ester;
[5-ethyl-4-[[(1-phenylmethyl)-1H-indazol-5-yl]amino]pyrrolo[2,1-f][1,2,4]triazin-6-yl]-carbamic acid, (2S)-2-pyrrolidinylmethyl ester;
[5-ethyl-4-[[(1-phenylmethyl)-1H-indazol-5-yl]amino]pyrrolo[2,1-f][1,2,4]triazin-6-yl]-carbamic acid, (3R)-3-morpholinylmethyl ester;
[5-ethyl-4-[[(1-phenylmethyl)-1H-indazol-5-yl]amino]pyrrolo[2,1-f][1,2,4]triazin-6-yl]-carbamic acid, 3-[(3S)-3-hydroxy-1-pyrrolidinyl]propyl ester;
[5-ethyl-4-[[(1-phenylmethyl)-1H-indazol-5-yl]amino]pyrrolo[2,1-f][1,2,4]triazin-6-yl]-carbamic acid, 3-[(3S)-3-hydroxy-1-piperidinyl]propyl ester;
[5-ethyl-4-[[(1-phenylmethyl)-1H-indazol-5-yl]amino]pyrrolo[2,1-f][1,2,4]triazin-6-yl]-carbamic acid, (3R)-3-pyrrolidinylmethyl ester;
[5-ethyl-4-[[(1-phenylmethyl)-1H-indazol-5-yl]amino]pyrrolo[2,1-f][1,2,4]triazin-6-yl]-carbamic acid, 3-[(3R)-3-hydroxy-1-pyrrolidinyl]propyl ester;
[5-ethyl-4-[[(1-phenylmethyl)-1H-indazol-5-yl]amino]pyrrolo[2,1-f][1,2,4]triazin-6-yl]-carbamic acid, [(2S)-1-methyl-2-pyrrolidinyl]methyl ester;
[5-ethyl-4-[[(1-phenylmethyl)-1H-indazol-5-yl]amino]pyrrolo[2,1-f][1,2,4]triazin-6-yl]-carbamic acid, (2S)-2-morpholinylmethyl ester;
[5-ethyl-4-[[(1-phenylmethyl)-1H-indazol-5-yl]amino]pyrrolo[2,1-f][1,2,4]triazin-6-yl]-carbamic acid, (3S)-3-pyrrolidinylmethyl ester;
[5-ethyl-4-[[(1-phenylmethyl)-1H-indazol-5-yl]amino]pyrrolo[2,1-f][1,2,4]triazin-6-yl]-carbamic acid, (2R)-2-morpholinylmethyl ester;
[5-ethyl-4-[[(1-phenylmethyl)-1H-indazol-5-yl]amino]pyrrolo[2,1-f][1,2,4]triazin-6-yl]-carbamic acid, [(3R)-1-methyl-3-pyrrolidinyl]methyl ester;
[5-ethyl-4-[[(1-phenylmethyl)-1H-indazol-5-yl]amino]pyrrolo[2,1-f][1,2,4]triazin-6-yl]-carbamic acid, trans-4-aminocyclohexyl ester;
[5-ethyl-4-[[(1-phenylmethyl)-1H-indazol-5-yl]amino]pyrrolo[2,1-f][1,2,4]triazin-6-yl]-carbamic acid, (3R)-3-piperidinyl ester;
[5-ethyl-4-[[(1-phenylmethyl)-1H-indazol-5-yl]amino]pyrrolo[2,1-f][1,2,4]triazin-6-yl]-carbamic acid, (3S)-3-piperidinyl ester;
[5-ethyl-4-[[(1-phenylmethyl)-1H-indazol-5-yl]amino]pyrrolo[2,1-f][1,2,4]triazin-6-yl]-carbamic acid, cis-4-aminocyclohexyl;
[5-ethyl-4-[[(1-phenylmethyl)-1H-indazol-5-yl]amino]pyrrolo[2,1-f][1,2,4]triazin-6-yl]-carbamic acid, (2R,4R)-2-(hydroxymethyl)-4-piperidinyl ester;
[5-ethyl-4-[[(1-phenylmethyl)-1H-indazol-5-yl]amino]pyrrolo[2,1-f][1,2,4]triazin-6-yl]-carbamic acid, (2S)-2-(hydroxymethyl)-4-piperidinyl ester;
[5-ethyl-4-[[(1-phenylmethyl)-1H-indazol-5-yl]amino]pyrrolo[2,1-f][1,2,4]triazin-6-yl]-carbamic acid, cis-4-(aminomethyl)cyclohexyl ester;
[5-ethyl-4-[[(1-phenylmethyl)-1H-indazol-5-yl]amino]pyrrolo[2,1-f][1,2,4]triazin-6-yl]-carbamic acid, cis-4-amino-4-methylcyclohexyl ester;
[5-ethyl-4-[[(1-phenylmethyl)-1H-indazol-5-yl]amino]pyrrolo[2,1-f][1,2,4]triazin-6-yl]-carbamic acid, [(2R,4R)-4-(hydroxy-2-piperidinyl]methylester;
[5-ethyl-4-[[(1-phenylmethyl)-1H-indazol-5-yl]amino]pyrrolo[2,1-f][1,2,4]triazin-6-yl]-carbamic acid, trans-4-(aminomethyl)cyclohexyl ester;
[5-ethyl-4-[[1-(2-oxazolylmethyl)-1H-indazol-5-yl]amino]pyrrolo[2,1-f][1,2,4]triazin-6-yl]-carbamic acid, (3S)-3-morpholinylmethyl ester;
[5-ethyl-4-[[1-(2-thienylmethyl)-1H-indazol-5-yl]amino]pyrrolo[2,1-f][1,2,4]triazin-6-yl]-carbamic acid, (3S)-3-morpholinylmethyl ester;
[5-ethyl-4-[[1-[(3-fluorophenyl)methyl]-1H-indazol-5-yl]amino]pyrrolo[2,1-f][1,2,4]triazin-6-yl]-carbamic acid, (3S)-3-morpholinylmethyl ester;
[5-ethyl-4-[[1-(4-thiazolylmethyl)-1H-indazol-5-yl]amino]pyrrolo[2,1-f][1,2,4]triazin-6-yl]-carbamic acid, (3S)-3-morpholinylmethyl ester;
[5-ethyl-4-[[1-(3-thienylmethyl)-1H-indazol-5-yl]amino]pyrrolo[2,1-f][1,2,4]triazin-6-yl]-carbamic acid, (3S)-3-morpholinylmethyl ester;
[5-ethyl-4-[[1-(2-pyridinylmethyl)-1H-indazol-5-yl]amino]pyrrolo[2,1-f][1,2,4]triazin-6-yl]-carbamic acid, (3S)-3-morpholinylmethyl ester;
[5-ethyl-4-[[1-(2-thiazolylmethyl)-1H-indazol-5-yl]amino]pyrrolo[2,1-f][1,2,4]triazin-6-yl]-carbamic acid, (3S)-3-morpholinylmethyl ester;
[5-ethyl-4-[[1-(3-pyridinylmethyl)-1H-indazol-5-yl]amino]pyrrolo[2,1-f][1,2,4]triazin-6-yl]-carbamic acid, (3S)-3-morpholinylmethyl ester;
[5-ethyl-4-[[1-(pyrazinylmethyl)-1H-indazol-5-yl]amino]pyrrolo[2,1-f][1,2,4]triazin-6-yl]-carbamic acid, (3S)-3-morpholinylmethyl ester;
[4-[[1-(3-fluorophenyl)methyl]-1H-indazol-5-ylamino]-5-methyl-pyrrolo[2,1-f][1,2,4]triazin-6-yl]-carbamic acid, trans-4-aminocyclohexyl ester;
[4-[[1-(3-fluorophenyl)methyl]-1H-indazol-5-ylamino]-5-methyl-pyrrolo[2,1-f][1,2,4]triazin-6-yl]-carbamic acid, (2R,4R)-2-(hydroxymethyl)-4-piperidinyl ester;
[4-[[1-(3-fluorophenyl)methyl]-1H-indazol-5-ylamino]-5-methyl-pyrrolo[2,1-f][1,2,4]triazin-6-yl]-carbamic acid, (2S,4S)-2-(hydroxymethyl)-4-piperidinyl ester;
[4-[[-1-(3-fluorophenyl)methyl]-1H-indazol-5-ylamino]-5-methyl-pyrrolo[2,1-f][1,2,4]-triazin-6-yl]-carbamic acid, cis-4-aminocyclohexyl ester;
[4-[[1-(3-fluorophenyl)methyl]-1H-indazol-5-ylamino]-5-methyl-pyrrolo[2,1-f][1,2,4]triazin-6-yl]-carbamic acid, cis-4-amino-4-methyl-cyclohexyl ester;
[4-[[1-(3-fluorophenyl)methyl]-1H-indazol-5-ylamino]-5-methyl-pyrrolo[2,1-f][1,2,4]triazin-6-yl]-carbamic acid, (2R)-2-aminopropyl ester;
[4-[[1-(3-fluorophenyl)methyl]-1H-indazol-5-ylamino]-5-methyl-pyrrolo[2,1-f][1,2,4]triazin-6-yl]-carbamic acid, (2S)-2-aminopropyl ester;
[4-[[1-(3-fluorophenyl)methyl]-1H-indazol-5-ylamino]-5-methyl-pyrrolo[2,1-f][1,2,4]triazin-6-yl]-carbamic acid, (3R)-3-morpholinylmethyl ester;
[4-[[1-(3-fluorophenyl)methyl]-1H-indazol-5-ylamino]-5-methyl-pyrrolo[2,1-f][1,2,4]triazin-6-yl]-carbamic acid, (3S)-3-morpholinylmethyl ester;
[4-[[1-(3-fluorophenyl)methyl]-1H-indazol-5-ylamino]-5-methyl-pyrrolo[2,1-f][1,2,4]triazin-6-yl]-carbamic acid, (3R)-3-piperidinyl ester;
[4-[[1-(3-fluorophenyl)methyl]-1H-indazol-5-ylamino]-5-methyl-pyrrolo[2,1-f][1,2,4]triazin-6-yl]-carbamic acid, (3S)-3-piperidinyl ester;
3-[[[[[4-[[1[(3-fluorophenyl)methyl]-1H-indazol-5-yl]amino]-5-methylpyrrolo[2,1-f][1,2,4]-triazin-6-yl]amino]carbonyl]oxy]methyl]-4-morpholinecarboxylic acid, (3S)-1,1-dimethylethyl ester;
[4-[[1-(3-fluorophenyl)methyl]-1H-indazol-5-ylamino]-5-methyl-pyrrolo[2,1-f][1,2,4]triazin-6-yl]-carbamic acid, 3-morpholinylmethyl ester; and
[4-[[1-(3-fluorophenyl)methyl]-1H-indazol-5-ylamino]-5-methyl-pyrrolo[2,1-f][1,2,4]triazin-6-yl]-carbamic acid, (3R)-3-morpholinylmethyl ester;
and pharmaceutically acceptable salts, solvates, hydrates, or prodrugs thereof.

In yet another embodiment, the indazolylpyrrolotriazine is a compound of Formula II:

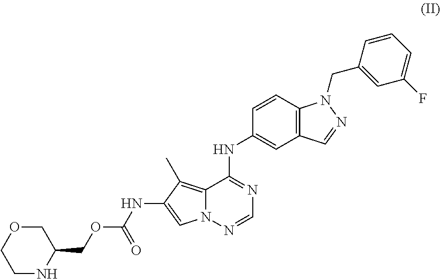

or a pharmaceutically acceptable salt, solvate, hydrate, or prodrug thereof.

The compound of Formula II is also known as AC480 or BMS-599626.

Further examples of indazolylpyrrolotriazines suitable for use in the methods provided herein are illustrated in U.S. Pat. Nos. 6,916,815; 7,102,001; and 7,148,220; and U.S. Pat. Pub. Nos. 2005/0209454 and 2006/0014741, the entirety of each of which is incorporated by reference herein.

The compound of Formula I can be prepared according to the methods described in U.S. Pat. Nos. 6,916,815; 7,102,001; and 7,148,220; and U.S. Pat. Pub. No. 2005/0209454 and 2006/0014741. The compound can be also synthesized according to other methods apparent to those of skill in the art based upon the teaching herein.

In one embodiment, the indazolylpyrrolotriazine used in the methods provided herein is a compound of Formula I, or a pharmaceutically acceptable solvate or hydrate thereof. In one embodiment, the compound of Formula I is a solid. In another embodiment, the compound of Formula I is a solid in an amorphous form. In yet another embodiment, the compound of Formula I is a solid in a crystalline form. In yet another embodiment, the compound of Formula I is a solvate. In yet another embodiment, the compound of Formula I is a hydrate. In yet another embodiment, the compound of Formula I is a monohydrate. In still another embodiment, the compound of Formula I is a monohydrate in a crystalline form.

In another embodiment, the indazolylpyrrolotriazine used in the methods provided herein is a free base of the compound of Formula II, or a pharmaceutically acceptable solvate or hydrate thereof. In one embodiment, the free base is a solid. In another embodiment, the free base is a solid in an amorphous form. In yet another embodiment, the free base is a solid in a crystalline form, including, but not limited to, the N-2 form. In yet another embodiment, the free base is a solid in the N-2 form. In yet another embodiment, the compound is a solvate. In yet another embodiment, the compound is a hydrate. In yet another embodiment, the compound is a monohydrate. In still another embodiment, the compound is a monohydrate in the H-1 form.

The compounds provided herein are intended to encompass all possible stereoisomers, unless a particular stereochemistry is specified. Where the indazolylpyrrolotriazine provided herein contains an alkenyl or alkenylene group, the compound may exist as one or a mixture of geometric cis/trans (or Z/E) isomers. Where structural isomers are interconvertible, the indazolylpyrrolotriazine may exist as a single tautomer or a mixture of tautomers. This can take the form of proton tautomerism in the indazolylpyrrolotriazine that contains, for example, an imino, keto, or oxime group; or so-called valence tautomerism in the compound that contain an aromatic moiety. It follows that a single indazolylpyrrolotriazine may exhibit more than one type of isomerism.

The indazolylpyrrolotriazines provided herein may be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of skill in the art will recognize that administration of a compound in its (R) form is equivalent, for compounds that undergo epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When the indazolylpyrrolotriazines provided herein contains an acidic or basic moiety, it may also be provided as a pharmaceutically acceptable salt (See, Berge et al., J. Pharm. Sci. 1977, 66, 1-19; and “Handbook of Pharmaceutical Salts, Properties, and Use,” Stahl and Wermuth, Ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, and sodium hydroxide; and organic bases, such as primary, secondary, tertiary, and quaternary, aliphatic, aromatic, heteroaryl, and heterocyclic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

In one embodiment, the compound suitable for use in the methods provided herein is a pharmaceutically acceptable salt of the compound of Formula I. In one embodiment, the salt is a solid. In another embodiment, the salt is a solid in an amorphous form. In yet another embodiment, the salt is a solid in a crystalline form, including, but not limited to, the N-1 form. In yet another embodiment, the salt is a solid in the N-1 form. In yet another embodiment, the salt is a hydrochloride salt. In yet another embodiment, the hydrochloride salt is in a crystalline form, including the N-1 form. In still another embodiment, the hydrochloride salt is a solid in the N-1 form.

In another embodiment, the compound suitable for use in the methods provided herein is a pharmaceutically acceptable salt of the compound of Formula II. In one embodiment, the salt is a solid. In another embodiment, the salt is a solid in an amorphous form. In yet another embodiment, the salt is a solid in a crystalline form, including, but not limited to, the N-1 form. In yet another embodiment, the salt is a solid in the N-1 form. In yet another embodiment, the salt is a hydrochloride salt. In yet another embodiment, the hydrochloride salt is in a crystalline form, including, but not limited to, the N-1 form. In still another embodiment, the hydrochloride salt is in the N-1 form.

The compound of Formula I in solid forms can be prepared according to the method described in U.S. Pat. Pub. No. 2006/0014741; or using other suitable methods known in the art.

The compound provided herein may also be provided as a prodrug, which is a functional derivative of the compound, for example, of Formula I or II and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; and Waller et al., Br. J. Clin. Pharmac. 1989, 28, 497-507.

### Pharmaceutical Compositions

Provided herein are pharmaceutical compositions comprising a compound provided herein, e.g., a compound of Formula I or II, as an active ingredient, including a single enantiomer, a mixture of enantiomers, or a mixture of diastereomers thereof; or a pharmaceutically acceptable salt, solvate, hydrate, or prodrug thereof, in combination with a pharmaceutically acceptable vehicle, carrier, diluent, or excipient, or a mixture thereof.

The compounds provided herein may be administered alone, or in combination with one or more other compounds or other active agents provided herein. The pharmaceutical compositions that comprise a compound provided herein, e.g., a compound of Formula I or II, as an active ingredient, including a single enantiomer, a mixture of enantiomers, or a mixture of diastereomers thereof; or a pharmaceutically acceptable salt, solvate, hydrate, or prodrug thereof; may be formulated in various dosage forms for oral, parenteral, and topical administration. The pharmaceutical compositions may also be formulated as modified release dosage forms, including delayed-, extended-, prolonged-, sustained-, pulsatile-, controlled-, accelerated-, fast-, targeted-, programmed-release, and gastric retention dosage forms. These dosage forms can be prepared according to conventional methods and techniques known to those skilled in the art (See, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2003; Vol. 126).

In one embodiment, the pharmaceutical compositions are provided in a dosage form for oral administration, which comprise a compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; and one or more pharmaceutically acceptable excipients or carriers.

In another embodiment, the pharmaceutical compositions are provided in a dosage form for parenteral administration, which comprise a compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; and one or more pharmaceutically acceptable excipients or carriers.

In yet another embodiment, the pharmaceutical compositions are provided in a dosage form for topical administration, which comprise a compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; and one or more pharmaceutically acceptable excipients or carriers.

The pharmaceutical compositions provided herein may be provided in a unit-dosage or multiple-dosage form. A unit-dosage form, as used herein, refers to physically discrete a unit suitable for administration to a subject, e.g., a human and animal subject, and packaged individually as is known in the art. Each unit-dose contains a predetermined quantity of an active ingredient(s) sufficient to produce the desired therapeutic effect, in association with the required pharmaceutical carriers or excipients. Examples of a unit-dosage form include an ampoule, syringe, and individually packaged tablet and capsule. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pints or gallons.

The pharmaceutical compositions provided herein may be administered at once, or multiple times at intervals of time. It is understood that the precise dosage and duration of treatment may vary with the age; weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

### A. Oral Administration

The pharmaceutical compositions provided herein for oral administration can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In addition to the active ingredient(s), the pharmaceutical compositions can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The amount of a binder or filler in the pharmaceutical compositions provided herein varies upon the type of formulation, and is readily discernible to those of ordinary skill in the art. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The amount of a diluent in the pharmaceutical compositions provided herein varies upon the type of formulation, and is readily discernible to those of ordinary skill in the art.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of a disintegrant in the pharmaceutical compositions provided herein varies upon the type of formulation, and is readily discernible to those of ordinary skill in the art. The amount of a disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include, but are not limited to, colloidal silicon dioxide, CAB-O-SIL® (Cabot Co. of Boston, Mass.), and asbestos-free talc. Suitable coloring agents include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone. Suitable preservatives include, but are not limited to, glycerin, methyl and propylparaben, benzoic add, sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

It should be understood that many carriers and excipients may serve several functions, even within the same formulation.

The pharmaceutical compositions provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein for oral administration can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or polyalkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration can be also provided in the forms of liposomes, micelles, microspheres, or nanosystems. Micellar dosage forms can be prepared as described in U.S. Pat. No. 6,350,458.

The pharmaceutical compositions provided herein for oral administration can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein for oral administration can be formulated as immediate or modified release dosage forms, including delayed-, sustained, pulsed-, controlled, targeted-, and programmed-release forms.

### B. Parenteral Administration

The pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, infrasternal, intracranial, intramuscular, intrasynovial, intravesical, and subcutaneous administration.

The pharmaceutical compositions provided herein for parenteral administration can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, crybprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents are those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

When the pharmaceutical compositions provided herein are formulated for multiple dosage administration, the multiple dosage parenteral formulations must contain an antimicrobial agent at bacteriostatic or fungistatic concentrations. All parenteral formulations must be sterile, as known and practiced in the art.

In one embodiment, the pharmaceutical compositions for parenteral administration are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein for parenteral administration can be formulated as immediate or modified release dosage forms, including delayed-, sustained, pulsed-, controlled, targeted-, and programmed-release forms.

The pharmaceutical compositions provided herein for parenteral administration can be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include, but are not limited to, polymethylmethacrylate, polybutyl-methacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogels of esters of acrylic and methacrylic acid, collagen, cross-linked polyvinyl alcohol, and cross-linked partially hydrolyzed polyvinyl acetate.

Suitable outer polymeric membranes include but are not limited to, polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical compositions provided herein can be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration.

The pharmaceutical compositions provided herein can be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches. The topical formulation of the pharmaceutical compositions provided herein can also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical compositions can also be administered topically by electroporation, iontophoresis, phonophoresis, sonophoresis, or microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein can be provided in the forms of ointments, creams, and gels. Suitable ointment vehicles include oleaginous or hydrocarbon vehicles, including lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Suitable cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include, but are not limited to, crosslinked acrylic acid polymers, such as carbomers, carboxypolyalkylenes, and CARBOPOL®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein can be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, and hydrogels, such as polyvinyl alcohol, hydroxyethyl methacrylate, and polyacrylic acid;. Combinations of the various vehicles can also be used. Rectal and vaginal suppositories may be prepared by compressing or molding. The typical weight of a rectal and vaginal suppository is about 2 to about 3 g.

The pharmaceutical compositions provided herein can be administered ophthalmically in the forms of solutions, suspensions, ointments, emulsions, gel-forming solutions, powders for solutions, gels, ocular inserts, and implants.

The pharmaceutical compositions provided herein can be administered intranasally or by inhalation to the respiratory tract. The pharmaceutical compositions can be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions can also be provided as a dry powder for insufflation, alone or in combination with an inert carrier such as lactose or phospholipids; and nasal drops. For intranasal use, the powder can comprise a bioadhesive agent, including chitosan or cyclodextrin.

Solutions or suspensions for use in a pressurized container, pump, spray, atomizer, or nebulizer can be formulated to contain ethanol, aqueous ethanol, or a suitable alternative agent for dispersing, solubilizing, or extending release of the active ingredient provided herein; a propellant as solvent; and/or a surfactant, such as sorbitan trioleate, oleic acid, or an oligolactic acid.

The pharmaceutical compositions provided herein can be micronized to a size suitable for delivery by inhalation, such as about 50 micrometers or less, or about 10 micrometers or less. Particles of such sizes can be prepared using a comminuting method known to those skilled in the art, such as spiral jet milling, fluid bed jet milling, supercritical fluid processing to form nanoparticles, high pressure homogenization, or spray drying.

Capsules, blisters, and cartridges for use in an inhaler or insufflator can be formulated to contain a powder mix of the pharmaceutical compositions provided herein; a suitable powder base, such as lactose or starch; and a performance modifier, such as l-leucine, mannitol, or magnesium stearate. The lactose may be anhydrous or in the form of the monohydrate. Other suitable excipients or carriers include, but are not limited to, dextran, glucose, maltose, sorbitol, xylitol, fructose, sucrose, and trehalose. The pharmaceutical compositions provided herein for inhaled/intranasal administration can further comprise a suitable flavor, such as menthol and levomenthol; and/or sweeteners, such as saccharin and saccharin sodium.

The pharmaceutical compositions provided herein for topical administration can be formulated to be immediate release or modified release, including delayed-, sustained-, pulsed-, controlled-, targeted, and programmed release.

### D. Modified Release

The pharmaceutical compositions provided herein can be formulated as a modified release dosage form. As used herein, the term “modified release” refers to a dosage form in which the rate or place of release of the active ingredient(s) is different from that of an immediate dosage form when administered by the same route. Modified release dosage forms include, but are not limited to, delayed-, extended-, prolonged-, sustained-, pulsatile-, controlled-, accelerated-, fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos.: 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

### 1. Matrix Controlled Release Devices

The pharmaceutical compositions provided herein in a modified release dosage form can be fabricated using a matrix controlled release device known to those skilled in the art (see, Takada et al. in “Encyclopedia of Controlled Drug Delivery,” Vol. 2, Mathiowitz Ed., Wiley, 1999).

In certain embodiments, the pharmaceutical compositions provided herein in a modified release dosage form is formulated using an erodible matrix device, which is water-swellable, erodible, or soluble polymers, including, but not limited to, synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethyl hydroxyethyl cellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methyl methacrylate, ethyl methacrylate, ethylacrylate, (2-dimethylaminoethyl)methacrylate, and (trimethylaminoethyl)methacrylate chloride.

In certain embodiments, the pharmaceutical compositions provided herein are formulated with a non-erodible matrix device. The active ingredient(s) is dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinyl acetate copolymers, ethylene/propylene copolymers, ethylene/ethyl acrylate copolymers, vinyl chloride copolymers with vinyl acetate, vinylidene chloride, ethylene and propylene, ionomer polyethylene terephthalate, butyl rubbers, epichlorohydrin rubbers, ethylene/vinyl alcohol copolymer, ethylene/vinyl acetate/vinyl alcohol terpolymer, ethylene/vinyloxyethanol copolymer, polyvinyl chloride, plasticized nylon, plasticized polyethylene terephthalate, natural rubber, silicone rubbers, polydimethylsiloxanes, and silicone carbonate copolymers; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical compositions provided herein in a modified release dosage form can be prepared by methods known to those skilled in the art, including direct compression, dry or wet granulation followed by compression, and melt-granulation followed by compression.

### 2. Osmotic Controlled Release Devices

The pharmaceutical compositions provided herein in a modified release dosage form can be fabricated using an osmotic controlled release device, including, but not limited to, one-chamber system, two-chamber system, asymmetric membrane technology (AMT), and extruding core system (ECS). In general, such devices have at least two components: (a) a core which contains an active ingredient; and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents is water-swellable hydrophilic polymers, which are also referred to as “osmopolymers” and “hydrogels.” Suitable water-swellable hydrophilic polymers as osmotic agents include, but are not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates can be employed to influence how rapidly the active ingredient(s) is initially delivered from the dosage form. For example, amorphous sugars, such as MANNOGEM™ EZ (SPI Pharma, Lewes, Del.) can be used to provide faster delivery during the first couple or hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core can also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane can also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane can be formed post-coating by mechanical or laser drilling. Delivery port(s) can also be formed in situ by erosion of a plug of water-soluble material or by rupture of a thinner portion of the membrane over an indentation in the core. In addition, delivery ports can be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form can further comprise additional conventional excipients or carriers as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In certain embodiments, the pharmaceutical compositions provided herein are formulated as AMT controlled-release dosage form, which comprises an asymmetric osmotic membrane that coats a core comprising the active ingredient(s) and other pharmaceutically acceptable excipients or carriers. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), a hydroxylethyl cellulose, and other pharmaceutically acceptable excipients or carriers.

### 3. Multiparticulate Controlled Release Devices

The pharmaceutical compositions provided herein in a modified release dosage form can be fabricated as a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates can be made by the processes known to those skilled in the art, including wet-and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein can be blended with the pharmaceutical compositions to aid in processing and forming the multiparticulates. The resulting particles can themselves constitute the multiparticulate device or can be coated by various film-forming materials, such as enteric polymers, water-swellable, and water-soluble polymers. The multiparticulates can be further processed as a capsule or a tablet.

### 4. Targeted Delivery

The pharmaceutical compositions provided herein can also be formulated to be targeted to a particular tissue, receptor, or other area of the body of the subject to be treated, including liposome-, resealed erythrocyte-, and antibody-based delivery systems. Examples include, but are not limited to, those disclosed in U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

### Radiotherapies

The compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; as a radiosensitizer, can be used with various forms of radiation, including, but not limited to, X-rays, visible light, lasers, infrared, microwave, radio frequencies, ultraviolet radiation, and other electromagnetic radiation at various frequencies; and particle beams of neutrons, electrons, and protons.

The methods provided herein can be performed with any suitable radiotherapy, including, but not limited to, external beam radiotherapy, also known as teletherapy; sealed source radiotherapy, also known as brachytherapy; unsealed source radiotherapy; radioisotope therapy; and radioimmunotherapy.

In one embodiment, the radiotherapy is external radiation therapy. Examples of external radiation therapy include, but are not limited to, conventional external beam radiotherapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiotherapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiotherapy, e.g., continuous hyperfractionated accelerated radiotherapy (CHART), in which more than one treatment (fraction) of radiotherapy are given to a subject per day; and hypofractionated radiotherapy, in which larger doses of radiotherapy per fraction is given but fewer fractions.

In another embodiment, the radiotherapy is internal radiation therapy. Example of internal radiation therapy include, but are not limited to, interstitial, intracavitary, intraluminal, intravenously radiation therapy, and implant radiation therapy, such as implantation of radioactive beads, particles, or seeds. In one embodiment, the radiotherapy is sealed source radiotherapy. In another embodiment, the radiotherapy is unsealed source radiotherapy.

In yet another embodiment, the radiotherapy is radioisotope therapy or radioimmunotherapy, where the radiotherapy is performed by administering a radioisotope parenterally to a subject, e.g., by injecting to a subject a tumor-specific antibody-radioisotope conjugate. Suitable radioisotopes for radioisotope therapy or radioimmunotherapy include, but are not limited to, ⁷²As, ¹⁹⁸Au, ²⁰⁶Bi, ⁷⁷Br, ¹¹C, ¹⁴C, ⁴⁷Ca, ¹²⁹Ce, ¹³⁷Ce, ⁵⁵Co, ⁵⁶Co, ⁵⁷Co, ⁵⁸Co, ⁶⁰Co, ⁵¹Cr, ⁶¹Cu, ¹⁶⁹Er, ¹⁸F, ⁵²Fe, ⁵⁵Fe, ⁵⁹Fe, ⁶⁷Ga, ¹²³I, ¹²⁵I, ¹³¹I, ¹¹¹In, ¹⁹²Ir, ⁸¹Kr, ¹⁷⁷Lu, ⁵²Mg, ¹³N, ²²Na, ²⁴Na, ⁵⁷Ni, ¹⁵O, ³²P, ²⁰³Pb, ¹⁰³Pd , ⁸¹Rb, ⁷²Se, ⁷³Se, ⁷⁵Se, ¹⁵³Sm, ⁸⁹Sr, ⁹⁰Sr, T, ⁹⁹Tc, ²⁰¹Tl, ¹⁶⁷Tm, ⁹⁰Y, ⁶²Zn, and ¹³³Xe. Examples of reagents for radioisotope therapy and radioimmunotherapy include, but not limited to, metaiodobenzylguanidine, oral iodine-131, hormone-bound lutetium-177 and yttrium-90, ibritumomab tiuxetan, tositumomab iodine-131, radioactive glass or resins, and radioactive nanoparticles.

The choice of the radiation therapy can be determined by taking into consideration various factors, including, e.g., the type, size, and location of the tumor, the age, weight, and condition of the subject being treated. It is understood that the precise dose of the radiation and duration of treatment may vary with the age, weight, and condition of the subject being treated, and may be determined empirically using known testing protocols or by extrapolation from in vivo or in vitro test or diagnostic data. It is also understood that the total radiation dose required is often divided into two or more fractions, which are administered over an extended period of time. It is further understood that for any particular individual, specific dosage regimens should be adjusted over time according to the individual need and the professional judgment of the person administering or supervising the administration of the radiation.

In one embodiment, the total dose given in the radiotherapy is ranging from about 1 Gy to about 500 Gy, from about 10 Gy to about 250 Gy, or from about 10 Gy to about 100 Gy. In certain embodiments, the total dose is divided into fractions and each fraction can be the same or different. Each fraction ranges from about 0.5 Gy to about 50 Gy, from about 0.5 Gy to about 25 Gy, from about 0.5 Gy to about 10 Gy, from about 0.5 Gy to about 5 Gy, from about 0.5 Gy to about 4 Gy, from about 1 Gy to about 4 Gy, from about 0.5 Gy to about 3 Gy, from about 1 Gy to about 3 Gy, from about 0.5 Gy to about 2 Gy, or from about 1 Gy to about 2 Gy.

### Methods of Use

In one embodiment, provided herein is a method of radiosensitizing a tumor in a subject, comprising administering to the subject a radiosensitizing amount of the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof.

In another embodiment, provided herein is a method of treating a tumor in a subject, comprising the steps of (A) radiosensitizing the tumor by administering to the subject a radiosensitizing amount of the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; and (B) administering to the subject a therapeutically effective dose of radiation.

In yet another embodiment, the radiosensitizing step (A) and the irradiation step (B) in the method provided herein are repeated so that a plurality of doses of the indazolpyrrolotriazine and a plurality of doses of radiation are administered to the subject.

In yet another embodiment, the radiosensitizing step (A) and the irradiation step (B) in the method provided herein are repeated so that a plurality of doses of the indazolpyrrolotriazine and a plurality of doses of radiation are administered to the subject over an extended period of time, wherein the extended period of time is ranging from 1 day to about 12 months, from 2 days to about 6 months, from 3 days to about 5 months, from 3 days to about 4 months, from 3 days to about 12 weeks, from 3 days to about 10 weeks, from 3 days to about 8 weeks, from 3 days to about 6 weeks, from 3 days to about 5 weeks, from 3 days to about 4 weeks, from 3 days to about 3 weeks, from 3 days to about 2 weeks, from 3 days to about 10 days, from 3 days to about 7 days, or from 3 days to about 5 days.

In one embodiment, in the methods provided herein, the compound is administered to the subject in the amount sufficient to produce a dose enhancement ratio of at least about 1.1, at least about 1.2, at least about 1.3, at least about 1.4, at least about 1.5, at least about 1.6, at least about 1.7, at least about 1.8, at least about 1.9, or at least about 2. In another embodiment, the compound is administered to the subject in the amount sufficient to produce a dose enhancement ratio of no greater than about 5, about 10, about 20, about 50, or about 100.

In one embodiment, the amount of the compound administered is sufficient to produce a dose enhancement ratio of at least about 1.1. In another embodiment, the amount of the compound administered is sufficient to produce a dose enhancement ratio of at least about 1.2. In yet another embodiment, the amount of the compound administered is sufficient to produce a dose enhancement ratio of at least about 1.3. In yet another embodiment, the amount of the compound administered is sufficient to produce a dose enhancement ratio of at least about 1.4. In yet another embodiment, the amount of the compound administered is sufficient to produce a dose enhancement ratio of at least about 1.5. In yet another embodiment, the amount of the compound administered is sufficient to produce a dose enhancement ratio of at least about 1.6. In yet another embodiment, the amount of the compound administered is sufficient to produce a dose enhancement ratio of at least about 1.7. In yet another embodiment, the amount of the compound administered is sufficient to produce a dose enhancement ratio of at least about 1.8. In yet another embodiment, the amount of the compound administered is sufficient to produce a dose enhancement ratio of at least about 1.9. In still another embodiment, the amount of the compound administered is sufficient to produce a dose enhancement ratio of at least about 2.

In one embodiment, the amount of the compound administered is sufficient to produce a dose enhancement ratio of at least about 1.1 but not greater than 50. In another embodiment, the amount of the compound administered is sufficient to produce a dose enhancement ratio of at least about 1.2 but not greater than 50. In yet another embodiment, the amount of the compound administered is sufficient to produce a dose enhancement ratio of at least about 1.3 but not greater than 50. In yet another embodiment, the amount of the compound administered is sufficient to produce a dose enhancement ratio of at least about 1.4 but not greater than 50. In yet another embodiment, the amount of the compound administered is sufficient to produce a dose enhancement ratio of at least about 1.5 but not greater than 50. In yet another embodiment, the amount of the compound administered is sufficient to produce a dose enhancement ratio of at least about 1.6 but not greater than 50. In yet another embodiment, the amount of the compound administered is sufficient to produce a dose enhancement ratio of at least about 1.7 but not greater than 50. In yet another embodiment, the amount of the compound administered is sufficient to produce a dose enhancement ratio of at least about 1.8 but not greater than 50. In yet another embodiment, the amount of the compound administered is sufficient to produce a dose enhancement ratio of at least about 1.9 but not greater than 50. In still another embodiment, the amount of the compound administered is sufficient to produce a dose enhancement ratio of at least about 2 but not greater than 50.

In certain embodiments, the compound is administered to the subject in the radiosensitizing amount, which ranges from about 1 ng/mL to about 1 mg/mL, from about 1 μg to 500 μg, from about 100 μg to about 400 μg or from about 150 μg to about 300 μg.

In certain embodiments, the compound is administered to the subject in the radiosensitizing amount, which ranges from about 0.01 to about 1,000 mg/kg, from about 0.1 to about 500 mg/kg, from about 0.1 to about 250 mg/kg, or from about 0.1 to about 100 mg/kg. In one embodiment, the radiosensitizing amount is from about 0.01 to about 1,000 mg/kg. In another embodiment, the radiosensitizing amount is from about 0.1 to about 500 mg/kg. In yet another embodiment, the radiosensitizing amount is from about 0.1 to about 250 mg/kg. In still another embodiment, the radiosensitizing amount is from about 0.1 to about 100 mg/kg.

In certain embodiments, the compound is administered to the subject in the radiosensitizing amount, which ranges from about 0.01 to about 1,000 mg/kg/day, from about 0.1 to about 500 mg/kg/day, from about 0.1 to about 250 mg/kg/day, or from about 0.1 to about 100 mg/kg/day. In one embodiment, the radiosensitizing amount is from about 0.01 to about 1,000 mg/kg/day. In another embodiment, the radiosensitizing amount is from about 0.1 to about 500 mg/kg/day. In yet another embodiment, the radiosensitizing amount is from about 0.1 to about 250 mg/kg/day. In still another embodiment, the radiosensitizing amount is from about 0.1 to about 100 mg/kg/day.

The administered dose of the compound provided herein can also be expressed in units other than the unit “mg/kg/day.” For example, doses for parenteral administration can be expressed as mg/m²/day. One of ordinary skill in the art would readily know how to convert doses from mg/kg/day to mg/m²/day to given either the height or weight of a subject or both (See, www.fda.gov/cder/cancer/animalframe.htm). For example, a dose of 1 mg/kg/day for a 65 kg human is approximately equal to 38 mg/m²/day.

In one embodiment, the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; is administered daily in a single dose or divided doses, where the total daily dose ranges from about 100 mg to about 1,200 mg, from about 200 mg to about 1,200 mg, from about 200 mg to about 1,100 mg, from about 300 mg to about 1,100 mg, from about 300 mg to about 1,000 mg, from about 320 mg to about 1,000 mg, from about from about 320 mg to about 900 mg, from about 325 mg to about 900 mg, from about 350 mg to about 900 mg, from about 350 mg to about 800 mg, or from about 400 to about 900 mg.

In another embodiment, the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; is administered daily in a single dose or divided doses, where the total daily dose ranges from about 300 mg to about 800 mg, from about 320 to about 800 mg, from about 320 to about 700 mg, from about 325 to about 650 mg, from about 325 mg to about 600 mg, or from about 350 mg to about 600 mg.

In yet another embodiment, the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; is administered daily in a single dose or divided doses for a total daily dose from about 300 mg to about 600 mg or from about 320 mg to about 600 mg.

In one embodiment, the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; is administered daily in a single dose or divided doses for a total daily dose of at least 200 mg, at least 250 mg, at least 300 mg, at least 320 mg, at least 325 mg, at least 350 mg, or at least 400 mg.

In certain embodiments, the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; is administered daily in a single dose or divided doses for a total daily dose sufficient to achieve a plasma concentration of the compound at steady state ranging from about 0.005 μM to about 10 μM, from about 0.5 μM to about 8 μM, from about 1 μM to about 6 μM, from about 1 μM to about 4 μM, from about 1.4 μM to about 3.8 μM, or from about 1.5 μM to about 3.5 μM. As used herein, the term “plasma concentration at steady state” is the concentration reached after a period of administration of a compound. Once steady state is reached, there are minor peaks and troughs on the time dependent curve of the plasma concentration of the compound.

In certain embodiments, the subject is a mammal. In certain embodiments, the mammal is a human.

In one embodiment, the tumor is a solid tumor. In another embodiment, the tumor is cancer.

In certain embodiments, the cancer treatable with the methods provided herein includes, but is not limited to, (1) leukemias, including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome or a symptom thereof (such as anemia, thrombocytopenia, neutropenia, bicytopenia or pancytopenia), refractory anemia (RA), RA with ringed sideroblasts (RARS), RA with excess blasts (RAEB), RAEB in transformation (RAEB-T), preleukemia, and chronic myelomonocytic leukemia (CMML), (2) chronic leukemias, including, but not limited to, chronic myelocytic (granulocytic) leukemia, chronic lymphocytic leukemia, and hairy cell leukemia; (3) polycythemia vera; (4) lymphomas, including, but not limited to, Hodgkin's disease and non-Hodgkin's disease; (5) multiple myelomas, including, but not limited to, smoldering multiple myeloma, nonsecretory myeloma, osteosclerotic myeloma, plasma cell leukemia, solitary plasmacytoma, and extramedullary plasmacytoma; (6) Waldenström's macroglobulinemia; (7) monoclonal gammopathy of undetermined significance; (8) benign monoclonal gammopathy; (9) heavy chain disease; (10) bone and connective tissue sarcomas, including, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, metastatic cancers, neurilemmoma, rhabdomyosarcoma, and synovial sarcoma; (11) brain tumors, including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; (12) breast cancer, including, but not limited to, adenocarcinoma, lobular (small cell) carcinoma, intraductal carcinoma, medullary breast cancer, mucinous breast cancer, tubular breast cancer, papillary breast cancer, primary cancers, Paget's disease, and inflammatory breast cancer; (13) adrenal cancer, including, but not limited to, pheochromocytom and adrenocortical carcinoma; (14) thyroid cancer, including, but not limited to, papillary or follicular thyroid cancer, medullary thyroid cancer, and anaplastic thyroid cancer; (15) pancreatic cancer, including, but not limited to, insulinoma, gastrinoma, glucagonoma, vipoma, somatostatin-secreting tumor, and carcinoid or islet cell tumor; (16) pituitary cancer, including, but limited to, Cushing's disease, prolactin-secreting tumor, acromegaly, and diabetes insipius; (17) eye cancer, including, but not limited, to ocular melanoma such as iris melanoma, choroidal melanoma, and cilliary body melanoma, and retinoblastoma; (18) vaginal cancer, including, but not limited to, squamous cell carcinoma, adenocarcinoma, and melanoma; (19) vulvar cancer, including, but not limited to, squamous cell carcinoma, melanoma, adenocarcinoma, basal cell carcinoma, sarcoma, and Paget's disease; (20) cervical cancers, including, but not limited to, squamous cell carcinoma, and adenocarcinoma; (21) uterine cancer, including, but not limited to, endometrial carcinoma and uterine sarcoma; (22) ovarian cancer, including, but not limited to, ovarian epithelial carcinoma, borderline tumor, germ cell tumor, and stromal tumor; (23) esophageal cancer, including, but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; (24) stomach cancer, including, but not limited to, adenocarcinoma, fungating (polypoid), ulcerating, superficial spreading, diffusely spreading, malignant lymphoma, liposarcoma, fibrosarcoma, and carcinosarcoma; (25) colon cancer; (26) rectal cancer; (27) liver cancer, including, but not limited to, hepatocellular carcinoma and hepatoblastoma; (28) gallbladder cancer, including, but not limited to, adenocarcinoma; (29) cholangiocarcinomas, including, but not limited to, pappillary, nodular, and diffuse; (30) lung cancer, including, but not limited to, non-small cell lung cancer, squamous cell carcinoma (epidermoid carcinoma), adenocarcinoma, large-cell carcinoma, and small-cell lung cancer; (31) testicular cancer, including, but not limited to, germinal tumor, seminoma, anaplastic, classic (typical), spermatocytic, nonseminoma, embryonal carcinoma, teratoma carcinoma, and choriocarcinoma (yolk-sac tumor); (32) prostate cancer, including, but not limited to, adenocarcinoma, leiomyosarcoma, and rhabdomyosarcoma; (33) penal cancer; (34) oral cancer, including, but not limited to, squamous cell carcinoma; (3S) basal cancer; (36) salivary gland cancer, including, but not limited to, adenocarcinoma, mucoepidermoid carcinoma, and adenoidcystic carcinoma; (37) pharynx cancer, including, but not limited to, squamous cell cancer and verrucous; (38) skin cancer, including, but not limited to, basal cell carcinoma, squamous cell carcinoma and melanoma, superficial spreading melanoma, nodular melanoma, lentigo malignant melanoma, and acral lentiginous melanoma; (39) kidney cancer, including, but not limited to, renal cell cancer, adenocarcinoma, hypernephroma, fibrosarcoma, and transitional cell cancer (renal pelvis and/or uterer); (40) Wilms' tumor; (41) bladder cancer, including, but not limited to, transitional cell carcinoma, squamous cell cancer, adenocarcinoma, and carcinosarcoma; and other cancer, including, not limited to, myxosarcoma, osteogenic sarcoma, endotheliosarcoma, lymphangio-endotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, and papillary adenocarcinomas (See Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

In certain embodiments, the cancer that is treatable with the methods provided herein includes, but is not limited to, bladder cancer, breast cancer, cervical cancer, colon cancer (e.g., colorectal cancer), esophageal cancer, head and neck cancer, liver cancer, lung cancer (e.g., small cell and non-small cell lung cancers), melanoma, myeloma, neuroblastoma, ovarian cancer, pancreatic cancer, prostate cancer, renal cancer, sarcoma (e.g., osteosarcoma), skin cancer (e.g., squamous cell carcinoma), stomach cancer, testicular cancer, thyroid cancer, and uterine cancer.

In one embodiment, the cancer treatable with the methods provided herein is bladder cancer, squamous cell cancer, head & neck cancer, colorectal cancer, esophageal cancer, gastric cancer, gynecological cancer, pancreatic cancer, rectal cancer, breast cancer, prostate cancer, vulva cancer, skin cancer, brain cancer, genitourinary tract cancer, lymphatic system cancer, stomach cancer, larynx cancer, or lung cancer.

In another embodiment, the cancer treatable with the methods provided herein is bladder cancer, head & neck cancer, colorectal cancer, esophageal cancer, gastric cancer, pancreatic cancer, brain cancer, rectal cancer, breast cancer, or non-small cell lung cancer.

In certain embodiments, the cancer is a metastatic cancer, including, but not limited to, bladder cancer, breast cancer, cervical cancer, colon cancer (e.g., colorectal cancer), esophageal cancer, head and neck cancer, liver cancer, lung cancer (e.g., small cell and non-small cell lung cancers), melanoma, myeloma, neuroblastoma, ovarian cancer, pancreatic cancer, prostate cancer, renal cancer, sarcoma (e.g., osteosarcoma), skin cancer (e.g., squamous cell carcinoma), stomach cancer, testicular cancer, thyroid cancer, and uterine cancer.

In certain embodiments, the subject to be treated with one of the methods provided herein has not been treated with anticancer therapy prior to the administration of the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof. In certain embodiments, the subject to be treated with one of the methods provided herein has been treated with anticancer therapy prior to the administration of the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof.

In certain embodiments, the subject to be treated with one of the methods provided herein has not been treated with radiotherapy. In certain embodiments, the subject to be treated with one of the methods provided herein has been treated with radiotherapy.

The methods provided herein encompass treating a subject regardless of patient's age, although some diseases or disorders are more common in certain age groups. Further provided is a method for treating a subject who has undergone surgery in an attempt to treat the disease or condition at issue, as well as the one who have not. Because the subjects with cancer have heterogeneous clinical manifestations and varying clinical outcomes, the treatment given to a particular subject may vary, depending on his/her prognosis.

Depending on the disease to be treated and the subject's condition, the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. The compound provided herein may be formulated, alone or together, in suitable dosage unit with pharmaceutically acceptable excipients, carriers, adjuvants and vehicles, appropriate for each route of administration. In one embodiment, the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; is administered orally. In another embodiment, the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; is administered parenterally. In yet another embodiment, the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; is administered intravenously.

The compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; can be delivered as a single dose such as, e.g., a single bolus injection, or oral tablets or pills; or over time such as, e.g., continuous infusion over time or divided bolus doses over time. The compound in combination with radiation can be administered repetitively if necessary, for example, until the patient experiences stable disease or regression, or until the patient experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 2000, 92, 205-216. Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

The compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; can be administered once daily (QD), or divided into multiple daily doses such as twice daily (BID), three times daily (TID), and four times daily (QID). In addition, the administration can be continuous, i.e., every day, or intermittently. The term “intermittent” or “intermittently” as used herein is intended to mean stopping and starting at either regular or irregular intervals. For example, intermittent administration of the compound provided herein is administration for one to six days per week, administration in cycles (e.g., daily administration for two to eight consecutive weeks, then a rest period with no administration for up to one week), or administration on alternate days.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, the compound provided herein is administered once a day. In another embodiment, The compound provided herein is administered twice a day. In yet another embodiment, The compound provided herein is administered three times a day. In still another embodiment, the compound provided herein is administered four times a day.

In certain embodiments, the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; is administered once per day from one day to six months, from one week to three months, from one week to four weeks, from one week to three weeks, or from one week to two weeks. In certain embodiments, the compound provided herein is administered once per day for one week, two weeks, three weeks, or four weeks. In one embodiment, the compound provided herein is administered once per day for one week. In another embodiment, the compound provided herein is administered once per day for two weeks. In yet another embodiment, the compound provided herein is administered once per day for three weeks. In still another embodiment, the compound provided herein is administered once per day for four weeks.

In one embodiment, the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; is administered daily in a single dose or divided doses for four days, five days, six days, one week, eight days, nine days, ten days, eleven days, twelve days, thirteen days, two weeks, three weeks four weeks, five weeks or six weeks, prior to the administration of the first dose of the radiation.

In another embodiment, the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; is administered daily in a single dose or divided doses for one week prior to the administration of the first dose of the radiation.

In yet another embodiment, the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; is administered daily in a single dose or divided doses for two weeks prior to the administration of the first dose of the radiation.

In certain embodiments, the radiation are administered one to four times per day in fractions from one day to six months, from one week to six months, from one week to five months, from one week to four months, from one week to three months, from one week to four weeks, from one week to three weeks, or from one week to two weeks. In certain embodiments, the radiation are administered in fractions once per day for one week, two weeks, three weeks, four weeks, three months, four months, five months, or six months. In certain embodiments, the radiation are administered in fractions twice per day for one week, two weeks, three weeks, four weeks, three months, four months, five months, or six months. In certain embodiments, the radiation are administered in fractions three times per day for one week, two weeks, three weeks, four weeks, three months, four months, five months, or six months.

In certain embodiments, the total dose of the radiation is administered in the course of one week, two weeks, three weeks, four weeks, five weeks, five and a half weeks, six weeks six and a half weeks, seven weeks, seven and a half weeks, eight weeks, or eight and a half weeks. In one embodiment, the total dose of the radiation is administered in the course of seven weeks. In another embodiment, the total dose of the radiation is administered in the course of eight weeks.

In certain embodiments, the radiation is administered in fractions for a total dose of about 24 Gy to about 84 Gy, from about 44 Gy to about 75 Gy, from about 50 Gy to about 77 Gy, from about 42 Gy to about 50 Gy, from about 54 Gy to about 60 Gy, from about 54 Gy to about 60 Gy, from about 50 Gy to about 60 Gy, from about 45 Gy to about 54 Gy, from about 45 Gy to about 50 Gy, from about 45 Gy to about 51 Gy, from about 30 Gy to about 45 Gy, from about 24 Gy to about 36 Gy, from about 50 Gy to about 51 Gy or from about 60 Gy to about 66 Gy.

In one embodiment, the radiation in a course of treatment is administered in fractions for a total dose of 81.6 Gy. In another embodiment, the radiation is administered in fractions for a total dose of 80.5 Gy. In another embodiment, the radiation is administered in fractions for a total dose of 72 Gy. In yet another embodiment, the radiation is administered in fractions for a total dose of 70 Gy. In yet another embodiment, the radiation is administered in fractions for a total dose of 50 Gy. In yet another embodiment, the radiation is administered in fractions for a total dose of 42.5 Gy. In yet another embodiment, the radiation is administered in fractions for a total dose of 40 Gy. In yet another embodiment, the radiation is administered in fractions for a total dose of 36 Gy. In yet another embodiment, the radiation is administered in fractions for a total dose of 30 Gy. In yet another embodiment, the radiation is administered in fractions for a total dose of up to 83.8 Gy. In yet another embodiment, the radiation is administered in fractions for a total dose of up to 77.4 Gy. In another embodiment, the radiation is administered in fractions for a total dose of up to 74 Gy. In yet another embodiment, the radiation is administered in fractions for a total dose of at least 50 Gy. In yet another embodiment, the radiation is administered in fractions for a total dose of at least 54 Gy. In yet another embodiment, the radiation is administered in fractions for a total dose of at least 60 Gy. In still another embodiment, the radiation is administered in fractions for a total dose of at least 70 Gy.

In one embodiment, the radiation in a course of treatment is administered in fractions for a total of 25 fractions, 25 to 28 fractions, 30 fractions, 35 fractions, 37 fractions, 38 fractions, 40 fractions, 42 fractions, 43 fractions, 45 fractions, 47 fractions, 48 fractions, 60 fractions, 61 fractions, 62 fractions, 63 fractions, 64 fractions, or 70 fractions. In another embodiment, the radiation in a course of treatment is administered in fractions for a total of 35 fractions. In yet another embodiment, the radiation therapy in a course of treatment is administered in fractions for a total of 40 fractions. In still another embodiment, the radiation in a course of treatment is administered in fractions for a total of 35 fractions.

In one embodiment, the radiation in a course of treatment is administered in once-daily fractions totaling three fractions per week, four fractions per week, five fractions per week, six fraction per week, or seven fractions per week. In one embodiment, the radiation in a course of treatment is administered in once-daily fractions totaling five fractions per week. In another embodiment, the radiation in a course of treatment is administered in once-daily fractions totaling seven fractions per week.

In another embodiment, the radiation in a course of treatment is administered in twice-daily fractions totaling six fractions per week, eight fractions per week, ten fractions per week, twelve fractions per week, or fourteen fractions per week.

In yet another embodiment, the radiation in a course of treatment is administered in thrice-daily fractions totaling nine fractions per week, twelve fractions per week, fifteen fractions per week, eighteen fractions per week or twenty-one fractions per week.

In one embodiment, the radiation dose administered per fraction ranges from about 1.1 Gy to about 3 Gy, from about 1.6 Gy to about 2 Gy, or from about 1.8 Gy to about 2 Gy. In another embodiment, the radiation dose administered per fraction ranges from about 1.8 Gy to about 2 Gy. In yet another embodiment, the radiation dose administered per fraction is about 1.15 Gy. In yet another embodiment, the radiation dose administered per fraction is about 1.6 Gy. In yet another embodiment, the radiation dose administered per fraction is about 1.8 Gy. In yet another embodiment, the radiation dose administered per fraction is about 2 Gy. In yet another embodiment, the radiation dose administered per fraction is about 2.15 Gy. In still another embodiment, the radiation dose administered per fraction is about 2.66 Gy.

In one embodiment, the radiation is administered in one day as a single high dose. In another embodiment, a radiation dose of 15 Gy is administered in one day. In yet another embodiment, a radiation dose of 18 Gy is administered in one day. In still another embodiment, a radiation dose of 24 Gy is administered in one day.

In certain embodiments, the radiation and the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; are cyclically administered to a patient. Cycling therapy involves the administration of the radiation and the compound provided herein for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

Consequently, in one embodiment, the radiation and the compound provided herein are administered daily for one week, two weeks, three weeks, four weeks, five weeks, six weeks, eight weeks, ten weeks, fifteen weeks, or twenty weeks, followed by a rest period of about 1 day to about ten weeks. For example, the methods contemplate using cycling of one week, two weeks, three weeks, four weeks, five weeks, six weeks, eight weeks, ten weeks, fifteen weeks, or twenty weeks. In another embodiment, the radiation and the compound provided herein are administered daily for one week, two weeks, three weeks, four weeks, five weeks, or six weeks with a rest period of 1, 3, 5, 7, 9, 12, 14, 16, 18, 20, 22, 24, 26, 28, 29 or 30 days. In certain embodiments, the rest period is 14 days. In certain embodiments, the rest period is 28 days. In one embodiment, the rest period is a period that is sufficient for bone marrow recovery. The frequency, number and length of dosing cycles can be increased or decreased.

In certain embodiments, the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; is administered to the subject in step (A) prior to the irradiation step (B). In certain embodiments, the compound provided herein is administered in step (A) to the subject about 2 days, about 1 day, about 12 hrs, about 6 hrs, about 4 hrs, about 2 hrs, about 60 min, about 30 min, about 10 min before the administration of the radiation in step (B).

In certain embodiments, the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; is administered to the subject in step (A) concurrently with the administration of the radiation in step (B).

In certain embodiments, the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; is administered in step (A) to the subject after the administration of the radiation in step (B). In certain embodiments, the compound provided herein is administered in step (A) to the subject about 2 days, about 1 day, about 12 hrs, about 6 hrs, about 4 hrs, about 2 hrs, about 60 min, about 30 min, about 10 min after the administration of the radiation in step (B).

In one embodiment, the radiotherapy and the administration of the compound provided herein occurs sequentially, where the compound administration is followed by radiotherapy, where the two therapies do not overlap. In another embodiment, the radiation therapy and the administration of the compound occurs sequentially, where the compound administration is followed the next day by radiotherapy, where the two therapies do not overlap.

In another embodiment, the radiotherapy and the administration of the compound provided herein occurs concurrently, where the compound administration is followed by radiotherapy, where the compound continues to be administered during radiotherapy.

In each embodiment provided herein, the method may further comprise a diagnostic step for determining the expression level of HER1 protein on the cells of the tumor. In one embodiment, the diagnostic step is carried out prior to the administration of the compound and radiation. If the subject has a tumor with overexpressed HER1, the administration of the compound and radiation is then followed. In another embodiment, the diagnostic step is carried out during the course of the treatment. In yet another embodiment, the diagnostic step is carried out after the administration of the compound and radiation. In still another embodiment, the diagnostic step is carried out after the administration of the total dose of the radiation.

In each embodiment provided herein, the method may further comprise a diagnostic step for determining the expression level of HER2 protein on the cells of the tumor. In one embodiment, the diagnostic step is carried out prior to the administration of the compound and radiation. In another embodiment, the diagnostic step is carried out during the course of the treatment.

The methods provided herein may further comprise administering other therapeutic agents useful in the treatment and/or prevention of a disease described herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

In certain embodiments, each method provided herein may independently, further comprise the step of administering a second therapeutic agent. In one embodiment, the second therapeutic agent is an anticancer agent. In another embodiment, the anticancer agent is an antimetabolite, including, but not limited to, 5-fluoro uracil, gemcitabine, methotrexate, cytarabine (also known as cytosine arabinoside or Ara-C), HDAC (high dose cytarabine) and fludarabine. In yet another embodiment, the anticancer agent is an antimicrotubule agent, including, but not limited to, vinca alkaloids (e.g., vincristine and vinblastine) and taxanes (e.g., paclitaxel and docetaxel). In yet another embodiment, the anticancer agent is an alkylating agent, including, but not limited to, cyclophosphamide, melphalan, carmustine, and nitrosoureas (e.g., bischloroethylnitrosurea and hydroxyurea). In yet another embodiment, the anticancer agent is a platinum agent, including, but not limited to, cisplatin, carboplatin, oxaliplatin, satraplatin (JM-216), and CI-973. In yet another embodiment, the anticancer agent is an anthracycline, including, but not limited to, doxrubicin and daunorubicin. In yet another embodiment, the anticancer agent is an antitumor antibiotic, including, but not limited to, bleomycin, mitomycin, idarubicin, adriamycin, and daunomycin (also known as daunorubicin). In yet another embodiment, the anticancer agent is a topoisomerase inhibitor, e.g., etoposide and camptothecins. In yet another embodiment, the anticancer agent is selected from the group consisting of adriamycin, busulfan, cytarabine, cyclophosphamide, dexamethasone, fludarabine, fluorouracil, hydroxyurea, interferons, oblimersen, platinum derivatives, taxol, topotecan, and vincristine. In yet another embodiment, the anticancer agent is a monoclonal antibody, including, but not limited to cetuximab.

The route of administration of the compound provided herein is independent of the route of administration of a second therapy. In one embodiment, the compound provided herein is administered orally. In another embodiment, the compound provided herein is administered intravenously. Thus, in accordance with these embodiments, the compound provided herein is administered orally or intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In one embodiment, the compound provided herein and a second therapy are administered by the same mode of administration, orally or by IV. In another embodiment, the compound provided herein is administered by one mode of administration, e.g., by IV, whereas the second agent (an anticancer agent) is administered by another mode of administration, e.g., orally.

Other therapies or anticancer agents that may be used in combination with the compound provided herein include surgery, endocrine therapy, biologic response modifiers (e.g., interferons, interleukins, and tumor necrosis factor (TNF)), hyperthermia and cryotherapy, agents to attenuate any adverse effects (e.g., antiemetics), and other approved chemotherapeutic drugs, including, but not limited to, alkylating drugs (mechlorethamine, chlorambucil, cyclophosphamide, melphalan, and ifosfamide), antimetabolites (cytarabine (also known as cytosine arabinoside or Ara-C), HDAC (high dose cytarabine), and methotrexate), purine antagonists and pyrimidine antagonists (6-mercaptopurine, 5-fluorouracil, cytarbine, and gemcitabine), spindle poisons (vinblastine, vincristine, vinorelbine, and paclitaxel), podophyllotoxins (etoposide, irinotecan, and topotecan), antibiotics (daunorubicin, doxorubicin, bleomycin, and mitomycin), nitrosoureas (carmustine and lomustine), inorganic ions (cisplatin and carboplatin), enzymes (asparaginase), and hormones (tamoxifen, leuprolide, flutamide, and megestrol), imatinib, adriamycin, dexamethasone, and cyclophosphamide. For a more comprehensive discussion of updated cancer therapies; See, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

In one embodiment, provided herein is a method of radiosensitizing a cell, comprising contacting the cell with a radiosensitizing amount of the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof.

In another embodiment, provided herein is a method of inhibiting the growth of a cell, comprising the steps of (A) radiosensitizing the cell by contacting the cell with a radiosensitizing amount of the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; and (B) exposing the cell to a dose of radiation sufficient to inhibit the growth of the cell.

In yet another embodiment, the radiosensitizing step (A) and the irradiation step (B) are repeated so that the cell is exposed to a plurality of doses of the indazolpyrrolotriazine and a plurality of doses of the radiation.

In yet another embodiment, the radiosensitizing step (A) and the irradiation step (B) are repeated so that the cell is exposed to a plurality of doses of the indazolpyrrolotriazine and a plurality of doses of the radiation over an extended period of time, wherein the extended period of time is ranging from 1 day to about 12 months, from 2 days to about 6 months, from 3 days to about 5 months, from 3 days to about 4 months, from 3 days to about 12 weeks, from 3 days to about 10 weeks, from 3 days to about 8 weeks, from 3 days to about 6 weeks, from 3 days to about 5 weeks, from 3 days to about 4 weeks, from 3 days to about 3 weeks, from 3 days to about 2 weeks, from 3 days to about 10 days, from 3 days to about 7 days, or from 3 days to about 5 days.

In one embodiment, the cell is contacted with the radiosensitizing amount of the compound provided herein sufficient to produce a dose enhancement ratio of at least about 1.1, at least about 1.2, at least about 1.3, at least about 1.4, at least about 1.5, at least about 1.6, at least about 1.7, at least about 1.8, at least about 1.9, or at least about 2. In another embodiment, the cell is contacted with the radiosensitizing amount of the compound provided herein sufficient to produce a dose enhancement ratio of no greater than about 5, about 10, about 20, about 50, or about 100. In certain embodiments, the dose enhancement ratio is measure at a surviving fraction of 0.25, 0.5, or 0.75.

In one embodiment, the cell is contacted with the radiosensitizing amount of the compound sufficient to produce a dose enhancement ratio of at least about 1.1. In another embodiment, the cell is contacted with the radiosensitizing amount of the compound sufficient to produce a dose enhancement ratio of at least about 1.2. In yet another embodiment, the cell is contacted with the radiosensitizing amount of the compound sufficient to produce a dose enhancement ratio of at least about 1.3. In yet another embodiment, the cell is contacted with the radiosensitizing amount of the compound sufficient to produce a dose enhancement ratio of at least about 1.4. In yet another embodiment, the cell is contacted with the radiosensitizing amount of the compound sufficient to produce a dose enhancement ratio of at least about 1.5. In yet another embodiment, the cell is contacted with the radiosensitizing amount of the compound sufficient to produce a dose enhancement ratio of at least about 1.6. In yet another embodiment, the cell is contacted with the radiosensitizing amount of the compound sufficient to produce a dose enhancement ratio of at least about 1.7. In yet another embodiment, the cell is contacted with the radiosensitizing amount of the compound sufficient to produce a dose enhancement ratio of at least about 1.8. In yet another embodiment, the cell is contacted with the radiosensitizing amount of the compound sufficient to produce a dose enhancement ratio of at least about 1.9. In still another embodiment, the cell is contacted with the radiosensitizing amount of the compound sufficient to produce a dose enhancement ratio of at least about 2. In certain embodiments, the dose enhancement ratio is measure at a surviving fraction of 0.25, 0.5, or 0.75.

In one embodiment, the cell is contacted with the radiosensitizing amount of the compound sufficient to produce a dose enhancement ratio of at least about 1.1 at a surviving fraction of 0.5. In another embodiment, the cell is contacted with the radiosensitizing amount of the compound sufficient to produce a dose enhancement ratio of at least about 1.2 at a surviving fraction of 0.5. In yet another embodiment, the cell is contacted with the radiosensitizing amount of the compound sufficient to produce a dose enhancement ratio of at least about 1.3 at a surviving fraction of 0.5. In yet another embodiment, the cell is contacted with the radiosensitizing amount of the compound sufficient to produce a dose enhancement ratio of at least about 1.4 at a surviving fraction of 0.5. In yet another embodiment, the cell is contacted with the radiosensitizing amount of the compound sufficient to produce a dose enhancement ratio of at least about 1.5 at a surviving fraction of 0.5. In yet another embodiment, the cell is contacted with the radiosensitizing amount of the compound sufficient to produce a dose enhancement ratio of at least about 1.6 at a surviving fraction of 0.5. In yet another embodiment, the cell is contacted with the radiosensitizing amount of the compound sufficient to produce a dose enhancement ratio of at least about 1.7 at a surviving fraction of 0.5. In yet another embodiment, the cell is contacted with the radiosensitizing amount of the compound sufficient to produce a dose enhancement ratio of at least about 1.8 at a surviving fraction of 0.5. In yet another embodiment, the cell is contacted with the radiosensitizing amount of the compound sufficient to produce a dose enhancement ratio of at least about 1.9 at a surviving fraction of 0.5. In still another embodiment, the cell is contacted with the radiosensitizing amount of the compound sufficient to produce a dose enhancement ratio of at least about 2 at a surviving fraction of 0.5.

In one embodiment, the cell is contacted with the radiosensitizing amount of the compound sufficient to produce a dose enhancement ratio of at least about 1.1 but not greater than 50. In another embodiment, the cell is contacted with the radiosensitizing amount of the compound sufficient to produce a dose enhancement ratio of at least about 1.2 but not greater than 50. In yet another embodiment, the cell is contacted with the radiosensitizing amount of the compound sufficient to produce a dose enhancement ratio of at least about 1.3 but not greater than 50. In yet another embodiment, the cell is contacted with the radiosensitizing amount of the compound sufficient to produce a dose enhancement ratio of at least about 1.4 but not greater than 50. In yet another embodiment, the cell is contacted with the radiosensitizing amount of the compound sufficient to produce a dose enhancement ratio of at least about 1.5 but not greater than 50. In yet another embodiment, the cell is contacted with the radiosensitizing amount of the compound sufficient to produce a dose enhancement ratio of at least about 1.6 but not greater than 50. In yet another embodiment, the cell is contacted with the radiosensitizing amount of the compound sufficient to produce a dose enhancement ratio of at least about 1.7 but not greater than 50. In yet another embodiment, the cell is contacted with the radiosensitizing amount of the compound sufficient to produce a dose enhancement ratio of at least about 1.8 but not greater than 50. In yet another embodiment, the cell is contacted with the radiosensitizing amount of the compound sufficient to produce a dose enhancement ratio of at least about 1.9 but not greater than 50. In still another embodiment, the cell is contacted with the radiosensitizing amount of the compound sufficient to produce a dose enhancement ratio of at least about 2 but not greater than 50. In certain embodiments, the dose enhancement ratio is measure at a surviving fraction of 0.25, 0.5, or 0.75.

In certain embodiments, the radiosensitizing amount of the compound provided herein ranges from about 1 nM to about 1 mM, from about 10 nM to about 10 μM, from about 20 nM to about 2 μM, or from about 100 nM to about 1 μM. In one embodiment, the radiosensitizing amount of the compound provided herein is 300 nM. In another embodiment, the radiosensitizing amount of the compound provided herein is 600 nM.

In certain embodiments, the cell is a mammalian cell. In certain embodiments, the mammal is a human cell. In certain embodiments, the cell is a tumor cell. In certain embodiments, the cell is mammalian tumor cell. In certain embodiments, the cell is a human tumor cell. In certain embodiments, the cell is a cancerous cell. In certain embodiments, the cell is mammalian cancerous cell. In certain embodiments, the cell is a human cancerous cell.

In certain embodiments, the cancerous cell that can be treated with the methods provided herein includes, but is not limited to, cells of bladder cancer, breast cancer, cervical cancer, colon cancer (e.g., colorectal cancer), esophageal cancer, head and neck cancer, liver cancer, lung cancer (e.g., small cell and non-small cell lung cancers), melanoma, myeloma, neuroblastoma, ovarian cancer, pancreatic cancer, prostate cancer, renal cancer, sarcoma (e.g., osteosarcoma), skin cancer (e.g., squamous cell carcinoma), stomach cancer, testicular cancer, thyroid cancer, and uterine cancer.

In one embodiment, the cell is a cell of bladder cancer, squamous cell cancer, head & neck cancer, colorectal cancer, esophageal cancer, gastric cancer, gynecological cancer, pancreatic cancer, rectal cancer, breast cancer, prostate cancer, vulva cancer, skin cancer, brain cancer, genitourinary tract cancer, lymphatic system cancer, stomach cancer, larynx cancer, or lung cancer.

In another embodiment, the cell is a cell of bladder cancer, head & neck cancer, colorectal cancer, esophageal cancer, gastric cancer, pancreatic cancer, brain cancer, rectal cancer, breast cancer, or non small cell lung cancer.

In certain embodiments, the cancerous cell is a metastatic cancerous cell, including, but not limited to, cells of bladder cancer, breast cancer, cervical cancer, colon cancer (e.g., colorectal cancer), esophageal cancer, head and neck cancer, liver cancer, lung cancer (e.g., small cell and non-small cell lung cancers), melanoma, myeloma, neuroblastoma, ovarian cancer, pancreatic cancer, prostate cancer, renal cancer, sarcoma (e.g., osteosarcoma), skin cancer (e.g., squamous cell carcinoma), stomach cancer, testicular cancer, thyroid cancer, and uterine cancer.

In certain embodiments, the cell is treated by contacting the cell in step (A) with the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; prior to the irradiation step (B). In certain embodiments, the cell is treated in step (A) with the compound provided herein, about 2 days, about 1 day, about 12 hrs, about 6 hrs, about 4 hrs, about 2 hrs, about 60 min, about 30 min, or about 10 min before the irradiation step (B).

In certain embodiments, the cell is treated in step (A) by contacting the cell with the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; concurrently with the irradiation step (B).

In certain embodiments, the cell is treated in step (A) by contacting the cell with the compound provided herein, e.g., a compound of Formula I or II, including a single enantiomer, a mixture of enantiomers, or a mixture of diastereomers thereof; or a pharmaceutically acceptable salt, solvate, hydrate, or prodrug thereof; after the irradiation step (B). In certain embodiments, the cell is treated in step (A) with the compound provided herein, about 2 days, about 1 day, about 12 hrs, about 6 hrs, about 4 hrs, about 2 hrs, about 60 min, about 30 min, or about 10 min after the irradiation step (B).

The inhibition of cell growth can be gauged by, e.g., counting the number of cells contacted with a compound of interest, comparing the cell proliferation with otherwise identical cells not contacted with the compound, or determining the size of the tumor that encompasses the cells. The number of cells, as well as the size of the cells, can be readily assessed using any method known in the art (e.g., trypan blue exclusion and cell counting, measuring incorporation of ³H-thymidine into nascent DNA in a cell).

### EXAMPLE

### Example 1

### Evaluation of AC480 Radiosensitization Effect in vitro

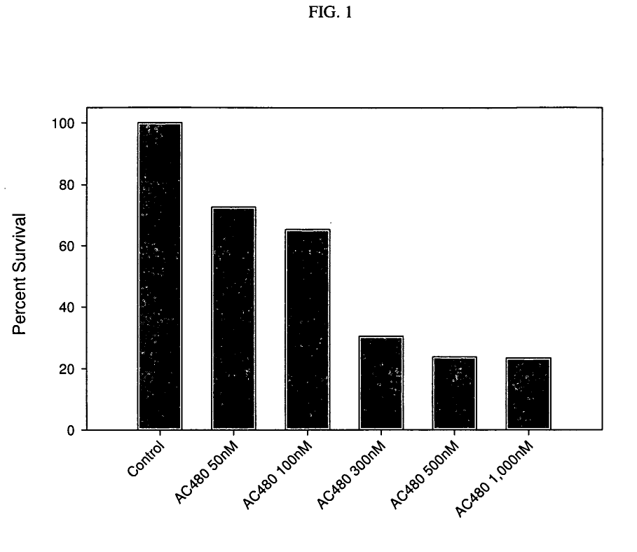

The radiosensitization effect of AC480 was evaluated in vitro using head neck squamous cell carcinoma cells, HNSCC-10, which express HER2 only and have no detectable amount of HER1 protein, and HN-5 cells, which express high levels of HER1 and HER2 proteins. AC480 has been shown to be cytotoxic to adenocarcinomas expressing HER1 and/or HER2 in salivary gland, breast, non-small cell lung, and gastric cancer cell lines (Wong et al., Clin. Cancer Res. 2006, 12, 6186-6193). AC480 was added to cell cultures that contained either HN-5 or HNSCC-10 cells 6 hrs before and kept in the medium for additional 66 hrs after irradiation. The cancerous cells were then irradiated with graded single doses of γ-radiation. At various predetermined time points after irradiation, the cells were assessed: (a) for clonogenic survival using clonogenic survival assays; (b) for apoptosis using aTUNEL assay; (c) for cell cycle distribution with FACS; and (d) for protein expression related to the cell cycle, apoptosis, and DNA repair, using Western blotting and immunoprecipitation methods. Results are shown in FIGS. 1 to 7.

a. Cell Proliferation Assay (MTT Assay)

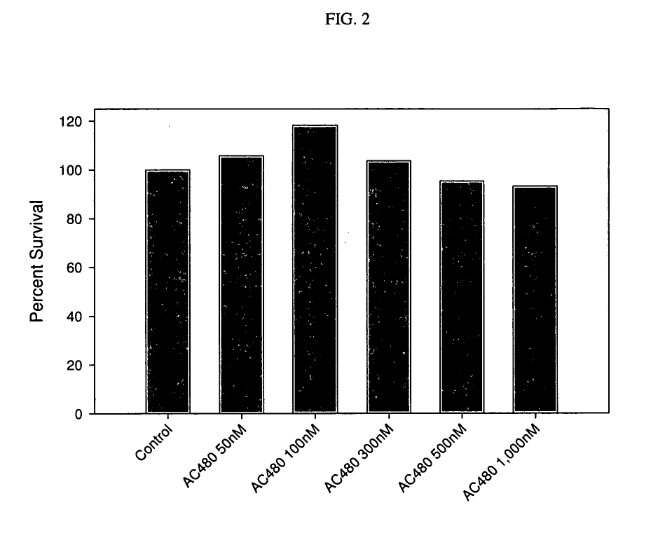

The effect of AC480 on cell proliferation of HN-5 and UMSCC-10 cells was determined using 3-[4,5-dimethylthiozol-2-yl]-2,5-diphenyltetrazolium bromide (MTT) assay. HN-5 and UMSCC-10 cells are plated in 96-well plates and treated with a series of concentrations of AC480. After 2 days, cells are stained with 3-[4,5-dimethylthiozol-2-yl]-2,5-diphenyltetrazolium bromide, and incubated for 4 hrs in a 37° C. incubator. The cells are then lysed in 150 μL of ethanol/DMSO mixture (1:1), and the absorbance read at 540 nm using a 96-well plate reader. Results are shown in FIGS. 1 and 2. AC480 (50 nM—1 μM, 72 hrs) inhibits the growth of HN-5 cells, but not UMSCC-10 cells.

b. Clonogenic Survival Assay

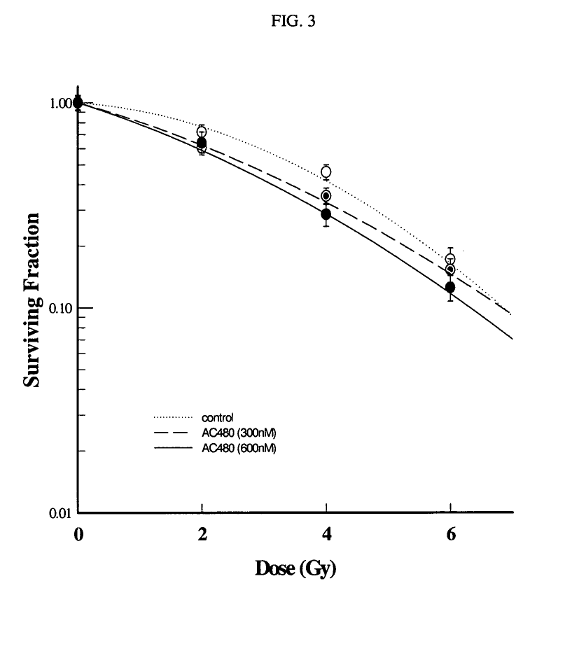

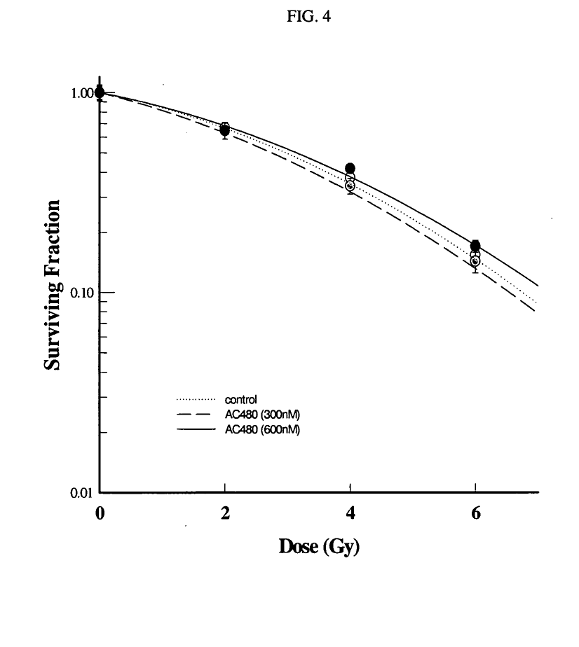

HN-5 or UMSCC-10 cells were counted and plated. Twenty-four hours later, the cells in culture were then exposed to AC480 at 300 or 600 nM for 6 hrs. The cells were then irradiated with graded doses at 2, 4, or 6 Gy of γ-rays using a ¹³⁷Cs source (3.7 Gy/min). Sixty-six hours later, culture media with AC480 were removed and fresh media were added. Media change was also done for untreated control cells at the same time. After 11 to 14 days of incubation, colonies were counted. Results are shown in FIGS. 3 and 4. AC480 significantly enhanced the radiosensitivity of HN-5, but not UMSCC-10 cells. The dose enhancement ratio at survival fraction of 0.5 was 1.30 for 300 nM and 1.43 for 600 nM of AC480. Dose enhancement ratio was calculated by dividing the radiation dose required to achieve a surviving fraction of 0.5 in the absence of AC480 with the radiation dose required to achieve the same surviving fraction in the presence of AC480 at a specified concentration.

c. TUNEL Assay

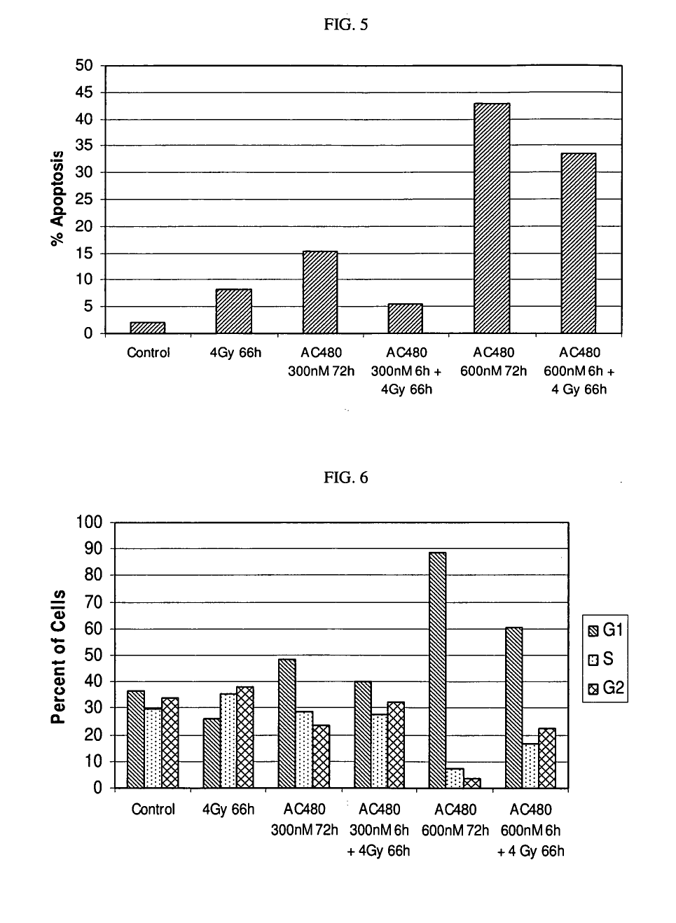

Apoptotic cells are detected by in-situ labeling of fragmented DNA with enzymatically labeled nucleotides using the TUNEL method as described in commercial protocols or by following the procedure described in Sgonc et al., Trends in Genetic 1994, 10, 41-42. AC480 increased radiation-induced apoptosis in HN-5 but not in UMSCC-10 cells (FIG. 5).

d. Cell-Cycle Analysis (FACS)

Cell cycle distribution is measured by determining the ploidy of cells by staining the cells with fluorescent dye and sorting by FACS using a commercially available kit. AC480 increased radiation-induced accumulation of HN-5 but not in UMSCC-10 cells in the G1 cell cycle phase (FIG. 6).

### e. γ-H2AX

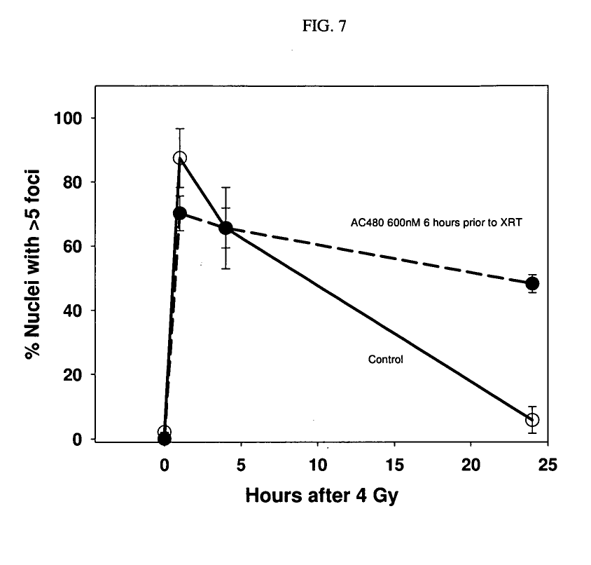

The extent of DNA repair is quantified by measuring the generation of nuclear foci at the site of DNA damage by phosphorylated H2AX (γ-H2AX), which is involved in the retention of repair and signaling factor complexes at sites of DNA double strand breaks. γ-H2AX is detected with antibodies to phosphorylated H2AX using traditional immunohistochemistry (See, e.g., Friesner et al., Mol. Biol. Cell. 2005, ⁻16, 2566-2576; and Dote et al., Clin. Cancer Res. 2005, 11, 4571-4579). AC480 prolonged the presence of γ-H2AX foci 24 hrs after radiation as assessed by immunocytochemistry (FIG. 7).

### Example 2

### Evaluation of AC480 Radiosensitization Effect in vivo

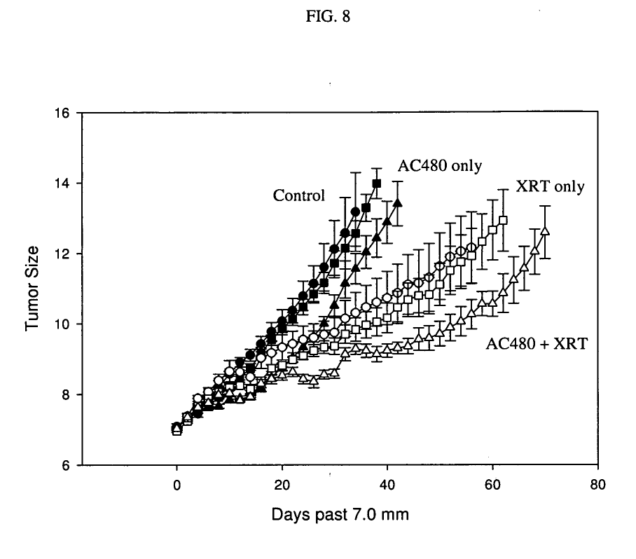

The effect of AC480 on radioresponse was assessed by tumor growth delay assay using HN-5 tumor xenografts generated in nude male mice. AC480 given before and during radiation was found to improve the radioresponse of HN-5 tumors in vivo, and the enhancement factor was 1.9 (FIG. 8).

Specifically, HN-5 cells (1×10⁶) were injected into the right leg of 3-4 month old nude male mice (nu-nu/Ncr) (SPF mice). When tumors grew to about 7.0 mm (6.8-7.3) in diameter, AC480 treatment was initiated (day 0) and continued once daily for 14 days. AC480 at a dosage of 120 mg/kg was dissolved in a mixture of propylene glycol and water (50:50) and administered via gavage in 0.1 mL per gram body weight. When the tumor reached 8.0 mm (7.8-8.4) in diameter (about 14-15 days after inoculation), fractionated radiotherapy (XRT) (5 Gy×5 consecutive days) was given. AC480 was administered 6 hrs prior to each fraction. Mice were treated with AC480 alone, XRT alone, combined, or received no treatment (control). Nine mice were in each group. Tumors were measured with calipers 3 times/week to determine tumor growth delay. Mice were sacrificed when tumors reached 14-15 mm.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A method of radiosensitizing a tumor in a subject, comprising administering to the subject a radiosensitizing amount of an indazolpyrrolotriazine of Formula I: 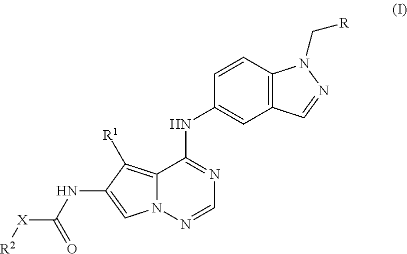
or an enantiomer, a mixture of enantiomers, or a mixture of two or more diastereomers thereof; or a pharmaceutically acceptable salt, solvate, hydrate, or prodrug thereof; wherein:
R is C₆₋₁₄ aryl or heterocyclyl;
R¹ is C₁₋₆ alkyl;
R² is hydrogen, C₁₋₆ alkyl, C₃₋₁₀ cycloalkyl, C₆₋₁₄ aryl, C₇₋₂₀ aralkyl, or heterocyclyl;
X is a bond, O, S, C(R³R⁴), or NR³; and
each R³ and R⁴ is independently hydrogen, C₁₋₆ alkyl, C₆₋₁₄ aryl, C₇₋₂₀ aralkyl, or heterocyclyl;
wherein each alkyl, cycloalkyl, aryl, aralkyl, and heterocyclyl is optionally substituted with one or more substituents Q selected from the group consisting of (a) cyano, halo, and nitro; (b) C₁₋₆ alkyl, C₂₋₆ alkenyl, C₂₋₆ alkynyl, C₃₋₁₀ cycloalkyl, C₆₋₁₄ aryl, heteroaryl, and heterocyclyl; and (c) —C(O)R^{a}, —C(O)OR^{a}, —C(O)NR^{b}R^{c}, —C(═NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{a}R^{d}, —NR^{a}C(O)R^{b}, —NR^{a}C(O)OR^{b}, —NR^{a}C(O)NR^{b}R^{c}, —NR^{a}C(═NR^{d})NR^{b}R^{c}, —NR^{a}S(O)R^{b}, —NR^{a}S(O)₂R^{b}, —NR^{a}S(O)NR^{b}R^{c}, —NR^{a}S(O)₂NR^{b}R^{c}, —SR^{a}, —S(O)R^{a}, and —S(O)₂R^{a}; wherein each R^{a}, R^{b}, R^{c}, and R^{d} is independently (i) hydrogen, C₁₋₆ alkyl, C₂₋₆ alkenyl, C₂₋₆ alkynyl, C₃₋₁₀ cycloalkyl, C₆₋₁₄ aryl, heteroaryl, or heterocyclyl; or (ii) R^{b} and R^{c} together with the N atom to which they are attached form heterocyclyl or heteroaryl.

2. A method of treating a tumor in a subject, comprising the steps of:
(A) radiosensitizing the tumor by administering to the subject a radiosensitizing amount of an indazolpyrrolotriazine of Formula I: 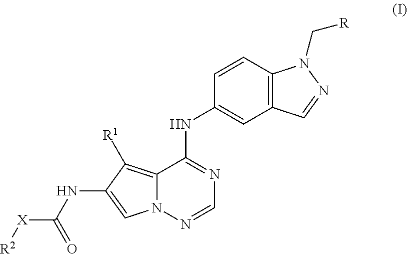
or an enantiomer, a mixture of enantiomers, or a mixture of two or more diastereomers thereof; or a pharmaceutically acceptable salt, solvate, hydrate, or prodrug thereof; wherein:
R is C₆₋₁₄ aryl or heterocyclyl;
R¹ is C₁₋₆ alkyl;
R² is hydrogen, C₁₋₆ alkyl, C₃₋₁₀ cycloalkyl, C₆₋₁₄ aryl, C₇₋₂₀ aralkyl, or heterocyclyl;
X is a bond, O, S, C(R³R⁴), or NR³; and
each R³ and R⁴ is independently hydrogen, C₁₋₆ alkyl, C₆₋₁₄ aryl, C₇₋₂₀ aralkyl, or heterocyclyl;
wherein each alkyl, cycloalkyl, aryl, aralkyl, and heterocyclyl is optionally substituted with one or more substituents Q selected from the group consisting of (a) cyano, halo, and nitro; (b) C₁₋₆ alkyl, C₂₋₆ alkenyl, C₂₋₆ alkynyl, C₃₋₁₀ cycloalkyl, C₆₋₁₄ aryl, heteroaryl, and heterocyclyl; and (c) —C(O)R^{a}, —C(O)OR^{a}, —C(O)NR^{b}R^{c}, —C(═NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{a}R^{d}, —NR^{a}C(O)R^{b}, —NR^{a}C(O)OR^{b}, —NR^{a}C(O)NR^{b}R^{c}, —NR^{a}C(═NR^{d})NR^{b}R^{c}, —NR^{a}S(O)R^{b}, —NR^{a}S(O)₂R^{b}, —NR^{a}S(O)NR^{b}R^{c}, —NR^{a}S(O)₂NR^{b}R^{c}, —SR^{a}, —S(O)R^{a}, and —S(O)₂R^{a}; wherein each R^{a}, R^{b}, R^{c}, and R^{d} is independently (i) hydrogen, C₁₋₆ alkyl, C₂₋₆ alkenyl, C₂₋₆ alkynyl, C₃₋₁₀ cycloalkyl, C₆₋₁₄ aryl, heteroaryl, or heterocyclyl;
or (ii) R^{b} and R^{c} together with the N atom to which they are attached form heterocyclyl or heteroaryl; and
(B) administering to the subject a therapeutically effective dose of radiation.

3. The method of claim 2, wherein the radiosensitizing step (A) is carried out before the irradiation step (B).

4. The method of claim 2, wherein the radiosensitizing step (A) is carried out concurrently with the irradiation step (B).

5. The method of claim 2, wherein the radiosensitizing step (A) is carried out after the irradiation step (B).

6. The method of claim 2, wherein the radiosensitizing step (A) and the irradiation step (B) are repeated so that a plurality of doses of the indazolpyrrolotriazine and a plurality of doses of the radiation are administered to the subject.

7. The method of claim 6, wherein the radiation dose is from about 0.5 Gy to about 4 Gy.

8. The method of claim 2, wherein the radiosensitizing step (A) and the irradiation step (B) are repeated so that a plurality of doses of the indazolpyrrolotriazine and a plurality of doses of the radiation are administered to the subject over an extended period of time, wherein the extended period of time is from about 1 day to about six months.

9. The method of claim 1, wherein the tumor is a solid tumor.

10. The method of claim 1, wherein the tumor is a malignant tumor.

11. The method of claim 10, wherein the malignant tumor is bladder cancer, squamous cell cancer, head & neck cancer, colorectal cancer, esophageal cancer, gastric cancer, gynecological cancer, pancreatic cancer, rectal cancer, breast cancer, prostate cancer, vulva cancer, skin cancer, brain cancer, genitourinary tract cancer, lymphatic system cancer, stomach cancer, larynx cancer, or lung cancer.

12. The method of claim 11, wherein the malignant tumor is bladder cancer, head & neck cancer, colorectal cancer, esophageal cancer, gastric cancer, pancreatic cancer, brain cancer, rectal cancer, breast cancer, or non small cell lung cancer.

13. The method of claim 1, wherein the tumor overexpresses HER1 protein.

14. The method of claim 13, wherein the tumor overexpresses HE R2 protein.

15. The method of claim 1, wherein the radiosensitizing amount of the indazolpyrrolotriazine is about 0.1 to about 250 mg/kg/day.

16. The method of claim 1, wherein the indazolpyrrolotriazine is administered orally.

17. The method of claim 1, wherein the indazolpyrrolotriazine is administered parenterally.

18. The method of claim 17, wherein the indazolpyrrolotriazine is administered intravenously.

19. The method of claim 1, further comprising diagnosing the subject to determine the presence of HER1 protein in the cells of the tumor.

20. A method of radiosensitizing a cell, comprising contacting the cell with a radiosensitizing amount of an indazolpyrrolotriazine of Formula I: 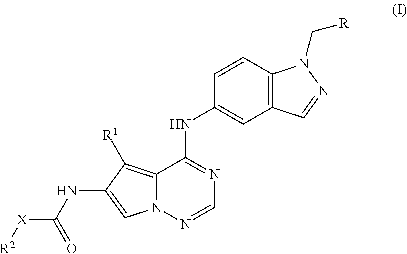
or an enantiomer, a mixture of enantiomers, or a mixture of two or more diastereomers thereof; or a pharmaceutically acceptable salt, solvate, hydrate, or prodrug thereof; wherein:
R is C₆₋₁₄ aryl or heterocyclyl;
R¹ is C₁₋₆ alkyl;
R² is hydrogen, C₁₋₆ alkyl, C₃₋₁₀ cycloalkyl, C₆₋₁₄ aryl, C₇₋₂₀ aralkyl, or heterocyclyl;
X is a bond, O, S, C(R³R⁴), or NR³; and
each R³ and R⁴ is independently hydrogen, C₁₋₆, alkyl, C₆₋₁₄ aryl, C₇₋₂₀ aralkyl, or heterocyclyl;
wherein each alkyl, cycloalkyl, aryl, aralkyl, and heterocyclyl is optionally substituted with one or more substituents Q selected from the group consisting of (a) cyano, halo, and nitro; (b) C₁₋₆ alkyl, C₂₋₆ alkenyl, C₂₋₆ alkynyl, C₃₋₁₀ cycloalkyl, C₆₋₁₄ aryl, heteroaryl, and heterocyclyl; and (c) —C(O)R^{a}, —C(O)OR^{a}, —C(O)NR^{b}R^{c}, —C(═NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{a}R^{d}, —NR^{a}C(O)R^{b}, —NR^{a}C(O)OR^{b}, —NR^{a}C(O)NR^{b}R^{c}, —NR^{a}C(═NR^{d})NR^{b}R^{c}, —NR^{a}S(O)R^{b}, —NR^{a}S(O)₂R^{b}, —NR^{a}S(O)NR^{b}R^{c}, —NR^{a}S(O)₂NR^{b}R^{c}, —SR^{a}, —S(O)R^{a}, and —S(O)₂R^{a}; wherein each R^{a}, R^{b}, R^{c}, and R^{d} is independently (i) hydrogen, C₁₋₆ alkyl, C₂₋₆ alkenyl, C₂₋₆ alkynyl, C₃₋₁₀ cycloalkyl, C₆₋₁₄ aryl, heteroaryl, or heterocyclyl;
or (ii) R^{b} and R^{c} together with the N atom to which they are attached form heterocyclyl or heteroaryl.

21. A method of inhibiting the growth of a cell, comprising the steps of:
(A) radiosensitizing the cell by contacting the cell with a radiosensitizing amount of an indazolpyrrolotriazine of Formula I: 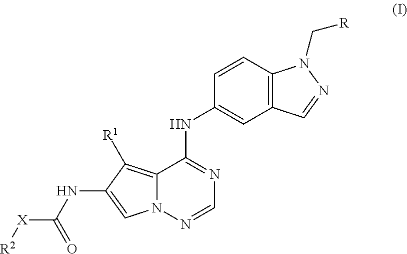
or an enantiomer, a mixture of enantiomers, or a mixture of two or more diastereomers thereof; or a pharmaceutically acceptable salt, solvate, hydrate, or prodrug thereof; wherein:
R is C₆₋₁₄ aryl or heterocyclyl;
R¹ is C₁₋₆ alkyl;
R² is hydrogen, C₁₋₆ alkyl, C₃₋₁₀ cycloalkyl, C₆₋₁₄ aryl, C₇₋₂₀ aralkyl, or heterocyclyl;
X is a bond, O, S, C(R³R⁴), or NR³; and
each R³ and R⁴ is independently hydrogen, C₁₋₆ alkyl, C₆₋₁₄ aryl, C₇₋₂₀ aralkyl, or heterocyclyl;
wherein each alkyl, cycloalkyl, aryl, aralkyl, and heterocyclyl is optionally substituted with one or more substituents Q selected from the group consisting of (a) cyano, halo, and nitro; (b) C₁₋₆ alkyl, C₂₋₆ alkenyl, C₂₋₆ alkynyl, C₃₋₁₀ cycloalkyl, C₆₋₁₄ aryl, heteroaryl, and heterocyclyl; and (c) —C(O)R^{a}, —C(O)OR^{a}, —C(O)NR^{b}R^{c}, —C(═NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{a}R^{d}, —NR^{a}C(O)R^{b}, —NR^{a}C(O)OR^{b}, —NR^{a}C(O)NR^{b}R^{c}, —NR^{a}C(═NR^{d})NR^{b}R^{c}, —NR^{a}S(O)R^{b}, —NR^{a}S(O)₂R^{b}, —NR^{a}S(O)NR^{b}R^{c}, —NR^{a}S(O)₂NR^{b}R^{c}, —SR^{a}, —S(O)R^{a}, and —S(O)₂R^{a}; wherein each R^{a}, R^{b}, R^{c}, and R^{d} is independently (i) hydrogen, C₁₋₆ alkyl, C₂₋₆ alkenyl, C₂₋₆ alkynyl, C₃₋₁₀ cycloalkyl, C₆₋₁₄ aryl, heteroaryl, or heterocyclyl; or (ii) R^{b} and R^{c} together with the N atom to which they are attached form heterocyclyl or heteroaryl; and
(B) exposing the cell to an effective fraction of radiation sufficient to inhibit the cell growth.

22. The method of claim 21, wherein the radiosensitizing step (A) is carried out prior to the irradiation step (B).

23. The method of claim 21, wherein the radiosensitizing step (A) is carried out concurrently with the irradiation step (B).

24. The method of claim 21, wherein the radiosensitizing step (A) is carried out after the irradiation step (B).

25. The method of claim 21, wherein the radiosensitizing step (A) and the irradiation step (B) are repeated so that the cell is exposed to a plurality of doses of the indazolpyrrolotriazine and a plurality of doses of the radiation over an extended period of time.

26. The method of claim 25, wherein each radiation dose is from about 0.5 Gy to about 4 Gy.

27. The method of claim 21, wherein the radiosensitizing step (A) and the irradiation step (B) are repeated so that the cell is exposed to a plurality of doses of the indazolpyrrolotriazine and a plurality of doses of the radiation over an extended period of time, wherein the extended period of time is from about 1 day to about six months.

28. The method of claim 20, wherein the cell is a tumor cell.

29. The method of claim 20, wherein the cell is a solid tumor cell.

30. The method of claim 20, wherein the cell is a cancerous cell.

31. The method of claim 30, wherein the cancerous cell is a cell of bladder cancer, squamous cell cancer, head & neck cancer, colorectal cancer, esophageal cancer, gastric cancer, gynecological cancer, pancreatic cancer, rectal cancer, breast cancer, prostate cancer, vulva cancer, skin cancer, brain cancer, genitourinary tract cancer, lymphatic system cancer, stomach cancer, larynx cancer, or lung cancer.

32. The method of claim 30, wherein the cancerous cell is a cell of bladder cancer, head & neck cancer, colorectal cancer, esophageal cancer, gastric cancer, pancreatic cancer, brain cancer, rectal cancer, breast cancer, or non small cell lung cancer.

33. The method of claim 20, wherein the cell overexpresses HER1 protein.

34. The method of claim 33, wherein the cell overexpresses HER2 protein.

35. The method of claim 20, wherein the radiosensitizing amount of the indazolpyrrolotriazine is from about 10 nM to about 10 μM.

36. The method of claim 1, wherein R is C₆₋₁₄ aryl, and R¹ is C₁₋₄ alkyl, each optionally substituted with one or more substituents Q.

37. The method of claim 1, wherein X is O, and R² is cycloalkyl or heterocyclyl, each optionally substituted with one or more substituents Q.

38. The method of claim 1, wherein the compound is selected from the group consisting of:
[5-ethyl-4-[[(1-phenylmethyl)-1H-indazol-5-yl]amino]pyrrolo[2,1-f][1,2,4]triazin-6-yl]-carbamic acid, (3S)-3-morpholinylmethyl ester;
[5-ethyl-4-[[(1-phenylmethyl)-1H-indazol-5-yl]amino]pyrrolo[2,1-f][1,2,4]triazin-6-yl]-carbamic acid, (2R)-2-pyrrolidinylmethyl ester;
[5-ethyl-4-[[(1-phenylmethyl)-1H-indazol-5-yl]amino]pyrrolo[2,1-f][1,2,4]triazin-6-yl]-carbamic acid, (2S)-2-pyrrolidinylmethyl ester;
[5-ethyl-4-[[(1-phenylmethyl)-1H-indazol-5-yl]amino]pyrrolo[2,1-f][1,2,4]triazin-6-yl]-carbamic acid, (3R)-3-morpholinylmethyl ester;
[5-ethyl-4-[[(1-phenylmethyl)-1H-indazol-5-yl]amino]pyrrolo[2,1-f][1,2,4]triazin-6-yl]-carbamic acid, 3-[(3S)-3-hydroxy-1-pyrrolidinyl]propyl ester;
[5-ethyl-4-[[(1-phenylmethyl)-1H-indazol-5 amino]pyrrolo[2,1-f][1,2,4]triazin-6-yl]-carbamic acid, 3-[(3S)-3-hydroxy-1-piperidinyl]propyl ester;
[5-ethyl-4-[[(1-phenylmethyl)-1H-indazol-5-yl]amino]pyrrolo[2,1-f][1,2,4]triazin-6-yl]-carbamic acid, (3R)-3-pyrrolidinylmethyl ester;
[5-ethyl-4-[[(1-phenylmethyl)-1H-indazol-5 amino]pyrrolo[2,1-f][1,2,4]triazin-6-yl]-carbamic acid, 3-[(3R)-3-hydroxy-1-pyrrolidinyl]propyl ester;
[5-ethyl-4-[[(1-phenylmethyl)-1H-indazol-5-yl]amino]pyrrolo[2,1-f][1,2,4]triazin-6-yl]-carbamic acid, [(2S)-1-methyl-2-pyrrolidinyl]methyl ester;
[5-ethyl-4-[[(1-phenylmethyl)-1H-indazol-5-yl]amino]pyrrolo[2,1-f][1,2,4]triazin-6-yl]-carbamic acid, (2S)-2-morpholinylmethyl ester;
[5-ethyl-4-[[(1-phenylmethyl)-1H-indazol-5-yl]amino]pyrrolo[2,1-f][1,2,4]triazin-6-yl]-carbamic acid, (3S)-3-pyrrolidinylmethyl ester;
[5-ethyl-4-[[(1-phenylmethyl)-1H-indazol-5-yl]amino]pyrrolo[2,1-f][1,2,4]triazin-6-yl]-carbamic acid, (2R)-2-morpholinylmethyl ester;
[5-ethyl-4-[[(1-phenylmethyl)-1H-indazol-5 amino]pyrrolo[2,1-f][1,2,4]triazin-6-yl)-carbamic acid, [(3R)-1-methyl-3-pyrrolidinyl]methyl ester;
[5-ethyl-4-[[(1-phenylmethyl)-1H-indazol-5-yl]amino]pyrrolo[2,1-f][1,2,4]triazin-6-yl]-carbamic acid, trans-4-aminocyclohexyl ester;
[5-ethyl-4-[[(1-phenylmethyl)-1H-indazol-5-yl]amino]pyrrolo[2,1-f][1,2,4]triazin-6-yl]-carbamic acid, (3R)-3-piperidinyl ester;
[5-ethyl-4-[[(1-phenylmethyl)-1H-indazol-5-yl]amino]pyrrolo[2,1-f][1,2,4]triazin-6-yl]-carbamic acid, (3S)-3-piperidinyl ester;
[5-ethyl-4-[[(1-phenylmethyl)-1H-indazol-5-yl]amino]pyrrolo[2,1-f][1,2,4]triazin-6-yl]-carbamic acid, cis-4-aminocyclohexyl;
[5-ethyl-4-[[(1-phenylmethyl)-1H-indazol-5-yl]amino]pyrrolo[2,1-f][1,2,4]triazin-6-yl]-carbamic acid, (2R,4R)-2-(hydroxymethyl)-4-piperidinyl ester;
[5-ethyl-4-[[(1-phenylmethyl)-1H-indazol-5-yl]amino]pyrrolo[2,1-f][1,2,4]triazin-6-yl]-carbamic acid, (2)-2-(hydroxymethyl)-4-piperidinyl ester;
[5-ethyl-4-[[(1-phenylmethyl)-1H-indazol-5-yl]amino]pyrrolo[2,1-f][1,2,4]triazin-6-yl]-carbamic acid, cis-4-(aminomethyl)cyclohexyl ester;
[5-ethyl-4-[[(1-phenylmethyl)-1H-indazol-5-yl]amino]pyrrolo[2,1-f][1,2,4]triazin-6-yl]-carbamic acid, cis-4-amino-4-methylcyclohexyl ester;
[5-ethyl-4-[[(1-phenylmethyl)-1H-indazol-5-yl]amino]pyrrolo[2,1-f][1,2,4]triazin-6-yl]carbamic acid, [(2R,4R)-4-(hydroxy-2-piperidinyl]methylester;
[5-ethyl-4-[[(1-phenylmethyl)-1H-indazol-5-yl]amino]pyrrolo[2,1-f][1,2,4]triazin-6-yl]carbamic acid, trans-4-(aminomethyl)cyclohexyl ester;
[5-ethyl-4-[[1-(2-oxazolylmethyl)-1H-indazol-5-yl]amino]pyrrolo[2,1-f][1,2,4]triazin-6-yl]-carbamic acid, (3S)-3-morpholinylmethyl ester;
[5-ethyl-4-[[1-(2-thienylmethyl)-1H-indazol-5-yl]amino]pyrrolo[2,1-f][1,2,4]triazin-6-yl]-carbamic acid, (3S)-3-morpholinylmethyl ester;
[5-ethyl-4-[[1-[(3-fluorophenyl)methyl]-1H-indazol-5-yl]amino]pyrrolo[2,1-f][1,2,4]triazin-6-yl]carbamic acid, (3S)-3-morpholinylmethyl ester;
[5-ethyl-4-[[1-(4-thiazolylmethyl)-1H-indazol-5-yl]amino]pyrrolo[2,1-f][1,2,4]triazin-6-yl]carbamic acid, (3S)-3-morpholinylmethyl ester;
[5-ethyl-4-[[1-(3-thienylmethyl)-1H-indazol-5-yl]amino]pyrrolo[2,1-f][1,2,4]triazin-6-yl]carbamic acid, (3S)-3-morpholinylmethyl ester;
[5-ethyl-4-[[1-(2-pyridinylmethyl)-1H-indazol-5-yl]amino]pyrrolo[2,1-f][1,2,4]triazin-6-yl]carbamic acid, (3S)-3-morpholinylmethyl ester;
[5-ethyl-4-[[1-(2-thiazolylmethyl)-1H-indazol-5-yl]amino]pyrrolo[2,1-f][1,2,4]triazin-6-yl]carbamic acid, (3S)-3-morpholinylmethyl ester;
[5-ethyl-4-[[1-(3-pyridinylmethyl)-1H-indazol-5-yl]amino]pyrrolo[2,1-f][1,2,4]triazin-6-yl]-carbamic acid, (3S)-3-morpholinylmethyl ester;
[5-ethyl-4-[[1-(pyrazinylmethyl)-1H-indazol-5-yl]amino]pyrrolo[2,1-f][1,2,4]triazin-6-yl]carbamic acid, (3S)-3-morpholinylmethyl ester;
[4-[[1-(3-fluorophenyl)methyl]-1H-indazol-5-ylamino]-5-methyl-pyrrolo[2,1-f][1,2,4]triazin-6-yl]carbamic acid, trans-4-aminocyclohexyl ester;
[4-[[1-(3-fluorophenyl)methyl]-1H-indazol-5-ylamino]-5-methyl-pyrrolo[2,1-f][1,2,4]triazin-6-yl]-carbamic acid, (2R,4R)-2-(hydroxymethyl)-4-piperidinyl ester;
[4-[[1-(3-fluorophenyl)methyl]-1H-indazol-5-ylamino]-5-methyl-pyrrolo[2,1-f][1,2,4]triazin-6-yl]-carbamic acid, (2S,4S)-2-(hydroxymethyl)-4-piperidinyl ester;
[4-[[-1-(3-fluorophenyl)methyl]-1H-indazol-5-ylamino]-5-methyl-pyrrolo[2,1-f][1,2,4]-triazin-6-yl]-carbamic acid, cis-4-aminocyclohexyl ester;
[4-[[1-(3-fluorophenyl)methyl]-1H-indazol-5-ylamino]-5-methyl-pyrrolo[2,1-f][1,2,4]triazin-6-yl]-carbamic acid, cis-4-amino-4-methyl-cyclohexyl ester;
[4-[[1-(3-fluorophenyl)methyl]-1H-indazol-5-ylamino]-5-methyl-pyrrolo[2,1-f][1,2,4]triazin-6-yl]-carbamic acid, (2R)-2-aminopropyl ester;
[4-[[1-(3-fluorophenyl)methyl]-1H-indazol-5-ylamino]-5-methyl-pyrrolo[2,1-f][1,2,4]triazin-6-yl]-carbamic acid, (2S)-2-aminopropyl ester;
[4-[[1-(3-fluorophenyl)methyl]-1H-indazol-5-ylamino]-5-methyl-pyrrolo[2,1-f][1,2,4]triazin-6-yl]-carbamic acid, (3S)-3-morpholinylmethyl ester;
[4-[[1-(3-fluorophenyl)methyl]-1H-indazol-5-ylamino]-5-methyl-pyrrolo[2,1-f][1,2,4]triazin-6-yl]-carbamic acid, (3R)-3-piperidinyl ester;
[4-[[1-(3-fluorophenyl)methyl]-1H-indazol-5-ylamino]-5-methyl-pyrrolo[2,1-f][1,2,4]triazin-6-yl]-carbamic acid, (3S)-3-piperidinyl ester;
3-[[[[[4-[[1[(3-fluorophenyl)methyl]-1H-indazol-5-yl]amino]-5-methylpyrrolo[2,1-f][1,2,4]-triazin-6-yl]amino]carbonyl]oxy]methyl]-4-morpholinecarboxylic acid, (3S)-1,1-dimethylethyl ester;
[4-[[1-(3-fluorophenyl)methyl]-1H-indazol-5-ylamino]-5-methyl-pyrrolo[2,1-f][1,2,4]triazin-6-yl]-carbamic acid, 3-morpholinylmethyl ester; and
[4-[[1-(3-fluorophenyl)methyl]-1H-indazol-5-ylamino]-5-methyl-pyrrolo[2,1-f][1,2,4]triazin-6-yl]-carbamic acid, (3R)-3-morpholinylmethyl ester;
and pharmaceutically acceptable salts, solvates, hydrates, and prodrugs thereof.

39. The method of claim 1, wherein the indazolpyrrolotriazine is 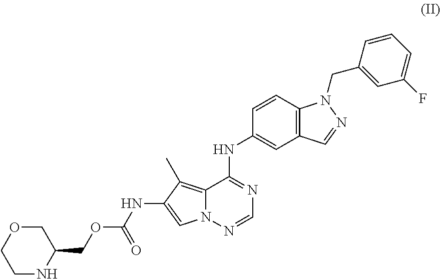
or a pharmaceutically acceptable salt, solvate, hydrate, or prodrug thereof.

40. The method of claim 1, wherein the radiosensitizing amount of the compound is sufficient to produce a dose enhancement ratio of at least about 1.2.

41. The method of claim 21, wherein the radiosensitizing amount of the compound is sufficient to produce a dose enhancement ratio of at least about 1.2 at a survival fraction of 0.5.
